SEQUENCE LISTING (43 sequences)

SEQ ID NO: 1 - protein, 10 aa, Artificial Sequence
  combinatorial random bacteriophage library
SQITLRLVSR

SEQ ID NO: 2 - protein, 12 aa, Artificial Sequence
  combinatorial random bacteriophage library
HRXHENSKQKGE

SEQ ID NO: 3 - protein, 12 aa, Artificial Sequence
  combinatorial random bacteriophage library
GKSQDGXRPWPE

SEQ ID NO: 4 - protein, 12 aa, Artificial Sequence
  combinatorial random bacteriophage library
ARXDAXACKNVN

SEQ ID NO: 5 - protein, 12 aa, Artificial Sequence
  combinatorial random bacteriophage library
EGPANXTQKHSM

SEQ ID NO: 6 - protein, 12 aa, Artificial Sequence
  combinatorial random bacteriophage library
RSGRDNQYWXMT

SEQ ID NO: 7 - protein, 16 aa, Artificial Sequence
  combinatorial random bacteriophage library
ALRRKAANVARTSAEQ

SEQ ID NO: 8 - protein, 12 aa, Artificial Sequence
  combinatorial random bacteriophage library
GPXGGRAQREAK

SEQ ID NO: 9 - protein, 12 aa, Artificial Sequence
  combinatorial random bacteriophage library
YAMPIXQTKFXY

SEQ ID NO: 10 - protein, 17 aa, Homo sapiens
SQISTTKRFEQELRLVS

SEQ ID NO: 11 - protein, 12 aa, Homo sapiens
WRQTQITLRGAD

SEQ ID NO: 12 - protein, 12 aa, Homo sapiens
GHGWRQTQITLR

SEQ ID NO: 13 - protein, 12 aa, Homo sapiens
GTATLRLVKRQV

SEQ ID NO: 14 - protein, 12 aa, Homo sapiens
LDGTATLRLVKR

SEQ ID NO: 15 - protein, 12 aa, Homo sapiens
LLHENSKQNGSA

SEQ ID NO: 16 - protein, 12 aa, Homo sapiens
LLRENSKQNGSA

SEQ ID NO: 17 - protein, 12 aa, Homo sapiens
VNEESSKQKGVL

SEQ ID NO: 18 - protein, 12 aa, Homo sapiens
LPREHANSKQEE

SEQ ID NO: 19 - protein, 12 aa, Homo sapiens
TELPREHANSKQ

SEQ ID NO: 20 - protein, 8 aa, Homo sapiens
ENSREKGD

SEQ ID NO: 21 - protein, 12 aa, Homo sapiens
  MISC_FEATURE (1)..(1): X is S or R
XIKAQDGGRPPL

SEQ ID NO: 22 - protein, 12 aa, Homo sapiens
KAQDGGRPPLIN

SEQ ID NO: 23 - protein, 12 aa, Homo sapiens
FASDACKNVTLH

SEQ ID NO: 24 - protein, 12 aa, Homo sapiens
ILIFASDACKNV

SEQ ID NO: 25 - protein, 12 aa, Homo sapiens
ARYASACKNADV

SEQ ID NO: 26 - protein, 12 aa, Homo sapiens
DSVARYASACKN

SEQ ID NO: 27 - protein, 16 aa, Homo sapiens
PANPHQKHAPVHSFSM

SEQ ID NO: 28 - protein, 12 aa, Homo sapiens
YFFVDNQYWRYD

SEQ ID NO: 29 - protein, 20 aa, Homo sapiens
QMVRVLRRTASRCAHVARTY

SEQ ID NO: 30 - protein, 20 aa, Homo sapiens
VLRRTASRCAHVARTYSIGR

SEQ ID NO: 31 - protein, 16 aa, Homo sapiens
SGETAANIARELAEQT

SEQ ID NO: 32 - protein, 16 aa, Homo sapiens
TAANIARELAEQTRNH

SEQ ID NO: 33 - protein, 20 aa, Homo sapiens
KRDRRGCFAVPMVHSTFLID

SEQ ID NO: 34 - protein, 20 aa, Homo sapiens
KRERRGCFAVPMVHSTFLID

SEQ ID NO: 35 - DNA, 30 bp, Artificial Sequence
  combinatorial random bacteriophage library
tctcagattactttacgcttagtctctcgc

SEQ ID NO: 36 - DNA, 36 bp, Artificial Sequence
  combinatorial random bacteriophage library
catcgctagcatgaaaattctaaacagaaaggtgaa

SEQ ID NO: 37 - DNA, 36 bp, Artificial Sequence
  combinatorial random bacteriophage library
ggtaaatctcaggatggttagcgcccttggcctgaa

SEQ ID NO: 38 - DNA, 36 bp, Artificial Sequence
  combinatorial random bacteriophage library
gcgaggtaggatgcctaggcttgtaagaatgtgaat

SEQ ID NO: 39 - DNA, 36 bp, Artificial Sequence
  combinatorial random bacteriophage library
gaggggcctgcgaattagacgcagaagcattcgatg

SEQ ID NO: 40 - DNA, 36 bp, Artificial Sequence
  combinatorial random bacteriophage library
cgttcgggtcgtgataatcagtattggtagatgacg

SEQ ID NO: 41 - DNA, 48 bp, Artificial Sequence
  combinatorial random bacteriophage library
gctttacgccgcaaagctgctaatgtcgctcgcacttctgctgaacag

SEQ ID NO: 42 - DNA, 36 bp, Artificial Sequence
  combinatorial random bacteriophage library
gggccgtagggggggagggcgcagagggaggcgaag

SEQ ID NO: 43 - DNA, 36 bp, Artificial Sequence
  combinatorial random bacteriophage library
tatgctatgcctatttagcagacgaagttttagtat

(19) United States
(12) Patent Application Publication
(10) Pub. No.: US 2011/0305682 A1
(43) Pub. Date: Dec. 15, 2011

(54) Compositions and Methods for Treating S. Pneumonia Infection

(75) Inventors: Yaffa Mizrachi-Nebenzahl, Beer Sheva (IL); Ron Dagan, Omer (IL)

(73) Assignee: Ben Gurion University of the Negev Research and Development Authority, Beer Sheva (IL)

(21) Appl. No.: 13/063,313

(22) PCT Filed: Sep. 10, 2009
(86) PCT No.: PCT/IL09/00882

§ 371 Date: Aug. 19, 2011

Related U.S. Application Data
(60) Provisional application No. 61/136,517, filed on Sep. 11, 2008.

(51) Int. Cl. A61K 38/45; C07K 7/06; A61K 38/10; A61P 29/00; A61K 38/48; A61P 31/04; A61P 11/02; A61P 11/08; C07K 7/08; A61K 38/08

(52) U.S. Cl. 424/94.5; 530/326; 530/327; 530/328; 514/2.6; 514/1.4; 424/94.67; 424/94.63

(57) ABSTRACT

The present invention relates to novel compounds that are useful for inhibition and prevention of pathogen cell adhesion and cell adhesion-mediated pathologies. This invention also relates to pharmaceutical formulations comprising these compounds and methods of using them for inhibition and prevention of pathogen cell adhesion and cell adhesion-mediated pathologies. The compounds and pharmaceutical compositions of this invention can be used as therapeutic or prophylactic agents. They are particularly well-suited for treatment of infectious diseases.

### FIELD OF INVENTION

The present invention relates to novel compounds that are useful for inhibition and prevention of pneumococcal infection and/or sepsis. The compounds and pharmaceutical compositions of this invention can be used as therapeutic or prophylactic agents.

### BACKGROUND OF THE INVENTION

Streptococcus pneumoniae is part of the flora of the human respiratory tract, and can cause invasive infections such as meningitis and sepsis. Mortality due to pneumococcal infection remains high all over the world, augmented by a widespread antibiotic resistance in many pneumococcal strains. The current polysaccharide-based vaccines elicit a strain-specific protection in children and the elderly, who are the main targets for pneumococcal infections. However, the available vaccines either do not elicit long-lasting protection or are limited in strain coverage. Development of new preventive and therapeutic intervention is hampered due to an incomplete understanding of pneumoccal pathogenesis.

The mucosal epithelial surfaces with their tight junctions constitute the first line of defense that prevents the entry of pathogens and their products. S. pneumoniae adhere to the nasopharyngeal mucosal cells, causing carriage without an overt inflammatory response. For clinical disease to occur, S. pneumoniae have to spread from the nasopharynx into the middle ear or the lungs or cross the mucosal epithelial cell layer and be deposited basally within the submucosa.

Pneumococci attachment to upper and lower respiratory tract cells is characterized by a broad area of contact, suggesting multiple receptor interactions. Molecules involved in adhesion, spread and invasion of S. pneumoniae include capsular polysaccharides, cell-wall peptidoglycan and surface proteins. S. pneumoniae-known adhesins include phosphorylcholine, which binds to the platelet activating factor receptor (PAF-R), Pav-A protein, which binds to the extracellular matrix component fibronectin, which in turn can bind epithelial cells through the integrin receptors, and the lipoproteins PsaA, Sma and Ami-AliaA/AliB, the target molecules of which are yet unclear. The choline binding protein A (SpsA/CbpA/PspC) is considered an invasin, since its binding either to the polymeric immunoglobulin receptor or to the secretory IgA facilitates the translocation of S. pneumoniae through the mucosal cell layer.

Adhesion of S. pneumoniae to host tissue is a multi-step process and a prerequisite to the development and progression of the disease. Not all adhesion-co-receptor interactions in S. pneumoniae have been identified, and an ideal prophylactic and therapeutic remains elusive.

### SUMMARY OF THE INVENTION

This invention provides, in one embodiment, an isolated polypeptide, wherein said polypeptide consists of an amino acid sequence as set forth in SEQ ID NOs: 10-14; 18-34.

This invention provides, in one embodiment, an isolated polypeptide, wherein said polypeptide consists of an amino acid sequence as set forth in SEQ ID NOs: 1-34.

This invention further provides, in one embodiment, a composition comprising an isolated polypeptide of this invention.

This invention further provides, in one embodiment, a method of treating or preventing S. pneumoniae infection in a subject, said method comprising administering to said subject a polypeptide of this invention, in an amount sufficient to inhibit or abrogate S. pneumoniae infection.

This invention further provides, in one embodiment, a method of treating or preventing sepsis caused by S. pneumoniae infection in a subject, said method comprising administering a polypeptide of this invention to said subject, in an amount sufficient to inhibit or abrogate sepsis in said subject.

### BRIEF DESCRIPTION OF THE FIGURES

FIG. 1. Autoradiograph showing identification of phages which bind to rGtS.

FIG. 2. Autoradiograph confirming phage binding to rGtS.

FIG. 3. Inhibition of adhesion of S. pneumoniae to lung epithelial cells by phages identified in FIG. 1, specifically 3A—phage G12; 3B—phage E9; 3C—phage F5; 3D—phage H12; 3E—phage H11; 3F—phage G3; 3G—phage A1; 3H—phage H11; and 3I—phage H6.

FIG. 4. Local alignment between a C1 phage polypeptide and SPOCK2.

### DETAILED DESCRIPTION OF THE INVENTION

This invention provides, inter alia, novel polypeptides, nucleic acids, compositions and methods for treatment or prevention of S. pneumoniae infection in a subject. The following are meant to provide materials, methods, and examples for illustrative purposes as a means of practicing/executing the present invention, and are not intended to be limiting.

Bacterial attachment is a prerequisite for disease development. The attached state of the bacteria is thought to promote nutrient uptake, allow bacterial multiplication and initiate a local immune response. Vaccine strategies target, inter alia, early events in pathogen invasion such as, for example, the activation of host cell proteins that activate signal transduction cascades and therefore may be attractive candidates targeted by vaccine design.

Glutamyl tRNA synthetase (GtS) is a cell wall-localized protein with age-dependent antigenicity. It is a class Ic synthetase. GtS has been found to be involved in the binding of S. pneumoniae to epithelial cells in culture. FACS analysis has confirmed surface localization of GtS on unencapsulated S. pneumoniae strains but not on the encapsulated strains. When S. pneumoniae adheres to epithelial cells, most of the polysaccharide capsule is shed. This shedding of the polysaccharide capsule may reveal cell wall-associated proteins which are otherwise masked.

Non-lectin proteins extracted from the S. pneumoniae cell wall have been found to be more effective in protecting mice from intraperitoneal inoculation than from intranasal inoculation. Such proteins, in some embodiments may be particularly useful in protecting a subject from sepsis mediated by S. pneumoniae.

Adhesion of S. pneumoniae to cells is thought to be mediated by a protein-protein interaction such as receptor binding. In one embodiment, phage display systems capable of displaying short peptides on the outer envelope of the phage represent good screens for potential polypeptides capable of binding to GtS, and thus represent an embodiment of a method contemplated in this invention.

In one embodiment, the present invention comprises polypeptides capable of inhibiting adhesion of S. pneumoniae to cells. In one embodiment, inhibition of adhesion of S. pneumoniae to cells inhibits and prevents pneumococcal infection and/or sepsis.

In one embodiment, the instant invention comprises peptide fragments of the polypeptides as herein described.

In one embodiment, a polypeptide of this invention has an amino acid sequence, which corresponds to or is homologous to SEQ ID NOs: 1-9, as shown in Table 1.

TABLE 1
Sequence ID NOs: 1-34.

| SEQ ID NO: | SEQUENCE: |
|---|---|
| 1 | SQITLRLVSR |
| 2 | HRXHENSKQKGE |
| 3 | GKSQDGXRPWPE |
| 4 | ARXDAXACKNVN |
| 5 | EGPANXTQKHSM |
| 6 | RSGRDNQYWXMT |
| 7 | ALRRKAANVARTSAEQ |
| 8 | GPXGGRAQREAK |
| 9 | YAMPIXQTKFXY |
| 10 | SQISTTKRFEQELRLVS |
| 11 | WRQTQITLRGAD |
| 12 | GHGWRQTQITLR |
| 13 | GTATLRLVKRQV |
| 14 | LDGTATLRLVKR |
| 15 | LLHENSKQNGSA |
| 16 | LLRENSKQNGSA |
| 17 | VNEESSKQKGVL |
| 18 | LPREHANSKQEE |
| 19 | TELPREHANSKQ |
| 20 | ENSREKGD |
| 21 | XIKAQDGGRPPL |
| 22 | KAQDGGRPPLIN |
| 23 | FASDACKNVTLH |
| 24 | ILIFASDACKNV |
| 25 | ARYASACKNADV |
| 26 | DSVARYASACKN |
| 27 | PANPHQKHAPVHSFSM |
| 28 | YFFVDNQYWRYD |
| 29 | QMVRVLRRTASRCAHVARTY |
| 30 | VLRRTASRCAHVARTYSIGR |
| 31 | SGETAANIARELAEQT |
| 32 | TAANIARELAEQTRNH |
| 33 | KRDRRGCFAVPMVHSTFLID |
| 34 | KRERRGCFAVPMVHSTFLID |

In one embodiment, the polypeptides of this invention include any polypeptide as herein described, any polypeptide which is homologous thereto, or in some embodiments, any polypeptide which specifically interacts with a polypeptide as herein described, which inhibits S. pneumoniae adhesion to and/or penetration of host cells.

In some embodiments, a polypeptide of this invention comprises a host cell protein or a fragment thereof, which interacts with S. pneumoniae, which is involved in bacterial adhesion and or invasion of the host cell. In some embodiments, a polypeptide of this invention interferes with the interaction, or in some embodiments, a polypeptide of this invention competes for binding to either member, or in some embodiments, both members of the cognate binding pair.

In some embodiments, the host cell protein or fragment thereof, which participates in adhesion to and/or invasion of the host cell has an amino acid sequence corresponding to, or homologous to SEQ ID NOs: 1-34 or 10-34.

In some embodiments, the host cell protein or fragment thereof, which participates in adhesion to and/or invasion of the host cell has an amino acid sequence corresponding to, or homologous to SEQ ID NOs: 1-34 or 10-34.

In some embodiments, this invention provides polynucleotides encoding the polypeptides of this invention. In one embodiment, the polynucleotides have a sequence corresponding to or homologous to that set forth in SEQ ID NOs: 35-43.

In some embodiments of this invention, inhibition of S. pneumoniae adhesion specifically to epithelial cells of the respiratory system may be mediated by any isolated polypeptide which is described herein, or fragments or homologues thereof. In one embodiment, isolated polypeptides will have a sequence corresponding to SEQ ID NOs. 1-34, or fragments or homologues thereof. In one embodiment, isolated polypeptides will have a sequence corresponding to SEQ ID NOs. 10-34 or 10-14; and 18-34, or fragments or homologues thereof.

In some embodiments, this invention provides a vector comprising a polynucleotide of this invention. In order to generate the nucleic acid constructs of the present invention disclosed herein, polynucleotide segments can be ligated into commercially available expression construct systems suitable for transforming bacterial cells and for directing the expression of the polypeptides of this invention, which in some embodiments, are specifically constructed such that multiple polypeptides are expressed as a fusion protein product within the transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides, and as such, encompass preferred embodiments of the present invention.

Suitable bacterial expression constructs for use with the present invention include, but are not limited to the pCAL, pUC, pET, pETBlue™ (Novagen), pBAD, pLEX, pTrcHis2, pSE280, pSE380, pSE420 (Invitrogen), pKK223-2 (Clontech), pTrc99A, pKK223-3, pRIT2T, pMC1871, pEZZ 18 (Pharmacia), pBluescript II SK (Stratagene), pALTER-Ex1, pALTER-Ex2, pGEMEX (Promega), pFivE (MBI), pQE (Qiagen) commercially available expression constructs, and their derivatives, and others known in the art. In some embodiments of the present invention the construct may also include, a virus, a plasmid, a bacmid, a phagemid, a cosmid, or a bacteriophage.

Nucleotide sequences are typically operably linked to, i.e., positioned, to ensure the functioning of an expression control sequence. These expression constructs are typically replicable in the cells either as episomes or as integral parts of the cell's chromosomal DNA, and may contain appropriate origins of replication for the respective prokaryotic strain employed for expression. Commonly, expression constructs contain selection markers, such as for example, tetracycline resistance, ampicillin resistance, kanamycin resistance or chlormaphenicol resistance, facilitating detection and/or selection of those bacterial cells transformed with the desired nucleic acid sequences (see, e.g., U.S. Pat. No. 4,704,362). These markers, however, are not exclusionary, and numerous others may be employed, as known to those skilled in the art. Indeed, in a preferred embodiment of the present invention expression constructs contain both positive and negative selection markers.

Similarly reporter genes may be incorporated within expression constructs to facilitate identification of transcribed products. Accordingly, in a preferred embodiment of the present invention, reporter genes utilized are selected from the group consisting of 3-galactosidase, chloramphenicol acetyl transferase, luciferase and a fluorescent protein

Prokaryotic promoter sequences regulate expression of the encoded polynucleotide sequences, and in some embodiments of the present invention, are operably linked to polynucleotides encoding the polypeptides of this invention. In additional embodiments of the present invention, these promoters are either constitutive or inducible, and provide a means of high and low levels of expression of the polypeptides of this invention, including in some embodiments, constructs specifically constructed to allow for a fusion gene of multiple polypeptides of the invention.

Many well-known bacterial promoters, including the T7 promoter system, the lactose promoter system, typtophan (Trp) promoter system, Trc/Tac Promoter Systems, beta-lactamase promoter system, tetA Promoter systems, arabiNOse regulated promoter system, Phage T5 Promoter, or a promoter system from phage lambda, may be employed, and others, as well, and comprise embodiments of the present invention. The promoters will typically control expression, optionally with an operator sequence and may include ribosome binding site sequences for example, for initiating and completing transcription and translation. According to additional embodiments, the vector may also contain expression control sequences, enhancers that may regulate the transcriptional activity of the promoter, appropriate restriction sites to facilitate cloning of inserts adjacent to the promoter and other necessary information processing sites, such as RNA splice sites, polyadenylation sites and transcription termination sequences as well as any other sequence which may facilitate the expression of the inserted nucleic acid.

In one embodiment, the present invention comprises antibodies to full length polypeptides or fragments thereof or multiple polypeptide fusion proteins of this invention, capable of inhibiting adhesion of S. pneumoniae to cells. In one embodiment, inhibition of adhesion of S. pneumoniae to cells by such antibodies inhibits and prevents pneumococcal infection and/or sepsis.

In another embodiment, the present invention comprises compositions comprising polynucleotides, vectors, polypeptides, peptide fragments and/or antibodies as herein described, capable of inhibiting S. pneumoniae adhesion to and/or invasion of host cells. In another embodiment, the present invention comprises methods of use of a polynucleotide, vector, polypeptide and/or fragment thereof as herein described and/or compositions comprising the same in treating, inhibiting or preventing pneumococcal infection and/or sepsis.

In one embodiment, the terms “isolated peptide” or “polypeptide” refers to a full length protein or in some embodiments a fragment understood as being less than the full-length portion of the native sequence of the protein in question, which in some embodiments may be a fusion polypeptide. Some polypeptides of this invention may comprise fusion polypeptides, wherein the polynucleotides of the invention are encoded by such fusion proteins. In one embodiment the fusion polypeptide encodes a selective marker. In another embodiment, the fusion polypeptide encodes a tag to facilitate ease of purification of said fusion polypeptide. In one embodiment, the fusion protein encodes a reporter as will be appreciated by one skilled in the art.

In one embodiment, the isolated peptide is a fragment of human laminin alpha 5 protein, for example, and in one embodiment, having a sequence corresponding to or homologous to that set forth in SEQ ID No: 10. In another embodiment, the isolated peptide is a fragment of nephronectin protein, for example, and in one embodiment, having a sequence corresponding to or homologous to that set forth in SEQ ID NOs: 11-12. In yet another embodiment, the isolated peptide is a fragment of SILV silver homolog protein, for example, and in one embodiment, having a sequence corresponding to or homologous to that set forth in SEQ ID NOs: 13-14. In yet another embodiment, the isolated peptide is a fragment of sparc/osteonectin or SPOCK2 protein, for example, and in one embodiment, having a sequence corresponding to or homologous to that set forth in SEQ ID NOs: 14-17. In yet another embodiment, the isolated peptide is a fragment of SPARC-likel protein, for example, and in one embodiment, having a sequence corresponding to or homologous to that set forth in SEQ ID NOs: 18-19. In yet another embodiment, the isolated peptide is a fragment of insulin-like growth factor 2 protein, for example, and in one embodiment, having a sequence corresponding to or homologous to that set forth in SEQ ID No: 20. In yet another embodiment, the isolated peptide is a fragment of cadherin protein, for example, and in one embodiment, having a sequence corresponding to or homologous to that set forth in SEQ ID NOs: 21-22. In yet another embodiment, the isolated peptide is a fragment of desmocollin protein, for example, and in one embodiment, having a sequence corresponding to or homologous to that set forth in SEQ ID NOs: 23-24. In yet another embodiment, the isolated peptide is a fragment of tectorin alpha protein, for example, and in one embodiment, having a sequence corresponding to or homologous to that set forth in SEQ ID NOs: 25-26. In yet another embodiment, the isolated peptide is a fragment of FRAS1-related extracellular matrix 1 protein, for example, and in one embodiment, having a sequence corresponding to or homologous to that set forth in SEQ ID No: 27. In yet another embodiment, the isolated peptide is a fragment of metalloprotease or metalloelastase protein, for example, and in one embodiment, having a sequence corresponding to or homologous to that set forth in SEQ ID No: 28. In yet another embodiment, the isolated peptide is a fragment of carboxylpeptidase Z or lactrophilin protein, for example, and in one embodiment, having a sequence corresponding to or homologous to that set forth in SEQ ID NOs: 29-32. In yet another embodiment, the isolated peptide is a fragment of glycosyltransferase 25 protein, for example, and in one embodiment, having a sequence corresponding to or homologous to that set forth in SEQ ID NOs: 33-34.

In one embodiment, the isolated peptide fragments of the subject invention or compositions containing said peptide fragments may be used to treat infection or sepsis caused by S. pneumoniae. In one embodiment, a polypeptide of this invention will comprise one having a sequence corresponding to, or homologous to that set forth in SEQ ID NOs: 1-9. In some embodiments, the polypeptides of this invention comprise polypeptides homologous thereto, which in some embodiments, may be derived from proteins described in NCBI's Genbank, having the following accession numbers: EAX07194.1; NP_{—}653259.3; AAU04438.1; CAI41411.1; AAH47713.1; BAB71253.1; EAW51489.1; BAC86230.1; EAW51488.1; P54296.1; NP_{—}003961.2; EAW51490.1; AAH52969.1; NP_{—}001003795.1; Q07954.1; NP_{—}002323.2; EAW96991.1; AAH21204.1; EAW79245.1; CAD38710.1; BAA92000.1; BAC04576.1; EAW96990.1; NP 443716.1; Q6UXI9.2; NP_{—}060732.2; BAD96815.1; CAB63718.1; EAX06194.1; NP 443715.1; AAH28353.1; Q9HBG6.1; EAW79246.1; AAH48252.1; AAY15057.1; EAW79250.1; NP 443711.1; AAG15428.1; AF244931_{—}1 BAB15060.1; NP_{—}149055.1; BAD18399.1; AAI32786.1; EAW70287.1; BAB69012.1; EAX06197.1; EAW70288.1; O15230; EAW75374.1; AAM12527.1; AF443072_{—}1; EAW75372.1; CAC22310.1; EAW75373.1; EAW70286.1; NP_{—}005551.3; BAB69013.1; EAW70285.1; BAC85701.1; EAW57945.1; NP_{—}009167.1; AAP29640.1; EAW89982.1; EAW96855.1; CAH72024.1; EAW52169.1; AAI53884.1; EAW96852.1; BAG06712.1; EAW60887.1; NP_{—}060110.3; Q8N815.2; AAD02890.1; EAW70029.1; NP 775749.1; NP_{—}001363.2; AAI17117.1; AAC51325.1; EAW89979.1; EAW89983.1; Q9NYC9.1; AAB19181.1; AAA18479.1; AAB31176.1; EAW89980.1; NP_{—}008859.1; EAW96854.1; AAA60121.1; EAW83288.1; AAC50293.1; NP_{—}037423.2; AAA35930.1; NP_{—}001092001.1; EAW57948.1; EAX03382.1; EAW82106.1; EAW76630.1; AAD12740.1; EAW93359.1; EAW90734.1; NP_{—}874369.1; AAC50656.1; EAW54441.1; EAW54442.1; BAA13404.2; BAF85017.1; NP_{—}055582.1; AAQ89280.1; NP_{—}001094891.2; EAW97859.1; EAW97858.1; EAW75156.1; NP_{—}004526.1; BAC11090.1; BAA74858.2; CAH18315.1; NP_{—}061923.2; CAE47751.1; 2PVS; BAF84181.1; 20XE; EAW49447.1; NP_{—}005387.2; BAA92588.1; EAW55311.1; EAW55310.1; NP_{—}002105.2; CAH73982.1; NP_{—}597677.2; P15822; DAA05331.1; BAF82544.1; EAW65792.1; EAW65795.1; NP_{—}060128.2; NP_{—}056199.2; EAW55948.1; AAK68113.1; BAA94476.1; BAA77335.1; NP_{—}000730.1; EAW55312.1; BAA77349.1; BAF82210.1; AAH35307.1; AAH73151.1; CAD30842.1; AAD00107.1; NP_{—}036587.2; BAD92035.1; P31249; NP_{—}008829.3; EAW73627.1; NP_{—}078887.2; CAH18700.1; NP_{—}001018099.1; AAH71685.1; CAH56220.1; CAH56197.1; AAH13684.1; BAB13948.1; AAH33721.1; NP_{—}004675.2; Q14515; AAH66647.1; EAW80740.1; EAX05991.1; NP_{—}055564.3; XP_{—}496003.3; NP_{—}001073883.2; XP_{—}946735.1; BAF85633.1; EAW78310.1; BAD92691.1; AA049801.1; NP_{—}001073960.1; AAD04169.1; AAD37716.1; AF146074_{—}1; BAA02807.2; XP_{—}932502.1; BAA25512.2; EAW80739.1; AAQ63404.1; Q9BVV6; BAB47438.1; NP_{—}940983.2; EAW67394.1; NP_{—}060242.2; NP_{—}064538.2; NP_{—}060484.2; XP 931193.1; CAC17724.1; Q4VX67.2; F75A4_HUMAN CAI95396.1; AAX93205.1; NP_{—}075044.2; CAC17723.1; EAW71010.1; NP_{—}056482.2; NP_{—}004837.1; Q5VVP1; AA088272; AAC62432.1; NP_{—}001003792.1; EAW69867.1; CAH71673.1; NP 443075.2; EAW69866.1; CAC44768.1; AAP75556.1; AAA60612.1; NP_{—}001092093.1; Q5VST9; AAC63910.1; NP_{—}001003793.1; 1O86; NP_{—}055298.2; BAF85478.1; EAW64405.1; 1O8A; 2OC2; 21UX; AAI17316.1; 2IUL; NP_{—}690043.1; EAW94319.1; EAW97933.1; EAW94315.1; EAW94312.1; EAW94316.1; BAD92208.1; AAH36375.1; NP_{—}000780.1; EAW94314.1; EAW94318.1; BAC03496.1; 1U71; EAW73429.1; NP_{—}055061.1; EAW73428.1; AAB04939.1; EAW64918.1; EAX08978.1; EAX08979.1; NP_{—}002262.3; EAW90389.1; EAW90391.1; NP_{—}036548.1; AAH45640.1; AAC14260.1; AAC51317.1; CAD97647.1; O00410.4; BAB63205.1; EAW94317.1; EAW64916.1; AAH88355.1; AAH10578.2; NP 689508.3; BAD96524.1; CAB65089.2; AAH12805.2; EAW83687.1; BAF83503.1; EAW51763.1; NP_{—}001036169.1; AAH53869.1; AAH48282.1; Q76MJ5; NP_{—}004061.3; EAW51764.1; NP_{—}000076.2; P51801; ABQ59030.1; EAW55802.1; ABQ59049.1; XP_{—}942747.2; NP_{—}150296.3; BAD18455.1; EAW51766.1; AAH00947.1; AAH20873.1; EAW51765.1; CAI16141.1; NP 694941.2; CAI42202.1; EAW88973.1; NP_{—}057699.2; EAW88975.1; AAI26216.1; NP_{—}003035.2; P48065; Q9NSD5; EAW88972.1; AAF64247.1; AAI46792.1; BAA74888.2; NP_{—}055826.1; BAA83024.2; CAD39038.1; AAH59382.1; NP_{—}001026884.3; Q9NUQ7; CAG33559.1; NP_{—}060829.1; EAX09063.1; EAW98036.1; EAW98034.1; EAW98032.1; EAW49678.1; XP_{—}942635.2; XP_{—}932070.2; BAD92172.1; AAH14420.1; NP_{—}004649.1; CAB66607.2; CAG28565.1; CAD38764.1; NP_{—}004302.1; O95294; NP 776152.1; CAB59184.1; ABM88171.1; ABM88170.1; BAF83002.1; NP_{—}004940.1; CAA40142.1; CAA40141.1; NP_{—}077740.1; EAW77923.1; BAD92649.1; NP_{—}002490.2; AAB17263.1; BAC03874.1; AAA61344.1; NP_{—}003374.3; AAA53684.1; NP_{—}001018066.1; 2OJ4; EAW87389.1; EAW87390.1; EAW87395.1; NP_{—}652760.2; AAH19039.3; NP 602299.1; AAM33254.1; AF490839_{—}1; AAL68829.1; AF463495_{—}1; AAM33253.1; AF490838_{—}1; BAB70766.1; AAH42555.1; AAM12641.1; AF493927_{—}1; AAM33255.1; AF490840_{—}1; AAM12655.1; AF493941_{—}1; AAH18072.2; NP_{—}570613.2; CAH70102.1; NP_{—}652759.3; NP_{—}066929.1; BAD92059.1; AAH10493.1; XP 944902.1; NP_{—}005413.2; EAW67518.1; EAW67517.1; O75443; EAW85906.1; EAX01090.1; EAW84934.1; EAW51360.1; BAD18617.1; AAX93261.1; AAA61218.1; AAA61216.1; AAL74416.1; AF439430_{—}1; AAA61215.1; AAN04473.1; AAH95448.1; AAY16985.1; NP_{—}783650.1; NP_{—}783653.1; NP_{—}000538.3; AAA61217.2; CAA35235.1; CA_A68467.1; AAW47397.1; NP_{—}922932.2; NP_{—}001091990.1; EAW68337.1; EAW68341.1; BAD96730.1; AAB84360.1; CAG33102.1; NP_{—}006401.3; Q9BUP3; AAC39694.1; EAW68340.1; NP_{—}001091993.1; Q9H324; EAW68907.1; NP 112219.2; AAA61219.1; AAG35563.1; AF163762_{—}1; XP_{—}001127680.1; EAW54951.1; CAM14447.1; AAI13996.1; BAB85549.1; NP_{—}542780.1; Q5H8C1; NP_{—}659403.4; EAW58688.1; EAW58690.1; EAW58689.1; BAD89015.1; NP_{—}056326.2; CAB55913.1; AAD27732.1; EAW67632.1; BAD96628.1; NP_{—}116237.1; NP_{—}001070.2; EAW72880.1; EAX01919.1; AAL38586.1; XP_{—}066859.5; EAX01918.1; EAX10939.1; BAC86413.1; AAH39039.1; NP_{—}997365.2; BAF83413.1; BAC04288.1; NP_{—}659802.1; NP_{—}997402.1; CAM330091; NP_{—}001008737.1; AAP57629.1; BAC03892.1; NP_{—}036584.1; BAD97173.1; BAB14332.1; EAX10737.1; Q8TBC5; AAH06205.2; NP_{—}076415.2; EAX08133.1; NP_{—}003975.1; 2EDJ; AAC39576.1; NP_{—}001035743.1; AAR84592.1; EAW67605.1; NP_{—}079388.3; BAF85375.1; AAH96276.1; Q2QGD7; NP_{—}009087.1; AAT96418.1; AAR02556.1; CAD82998.1; EAW51090.1; AAH22390.1; ABA71597.1; AAR84593.1; P15822; EAW55312.1; EAW55311.1; EAW55310.1; NP_{—}002105.2; CAH73982.1; BAC03805.1; BAC86316.1; BAC11205.1; AAH12729.2; EAX10287.1; BAF83734.1; EAX10289.1; ABF83430.1; AAH73859.1; Q9UKT9.1; CAC80429.1; EAW79361.1; NP_{—}899053.1; NP_{—}036613.2; EAW68889.1; AAR84587.1; CAC80428.1; CAC80431.1; CAC80427.1; NP 899051.1; NP 899055.1; NP 899052.1; CAA62189.1; EAW50807.1; XP_{—}936926.2; AAR84588.1; CAC80430.1; EAW67860.1; 2C6C; NP_{—}001014986.1; AAC83972.1; NP_{—}004467.1; BAC03426.1; NP_{—}060908.1; AAF04470.1; AF129536_{—}1; AAH39716.1; NP_{—}001898.1; EAW64510.1; EAW64508.1; EAW64512.1; EAW64509.1; EAW64511.1; NP_{—}031361.2; NP_{—}031363.2; Q9Y238; AAB36943.1; AAA58658.1; NP_{—}002417.2; AAC62830.1; NP_{—}055831.1; BAA25487.1; NP_{—}002855.2; EAW88588.1; EAW88589.1; AAI11757.1; CAA38255.1; EAW51938.1; AAF76236.1; B26524; CAA40890.1; CAA40889.1; NP_{—}113654.3; BAF94955.1; EAW99982.1; EAW94083.1; NP_{—}057700.3; AAH36816.1; CAB59415.1; AAY33378.1; S03496; BAF85134.1; EAW77820.1; AAL39001.1; AF111799_{—}1; AAD51919.2; AF137378_{—}1; Q9UKX5; NP_{—}001004439.1; 2ESV; CAC03858.1; CAE18185.1; CAB70676.1; CAH56735.1; CAC34111.1; EAW73904.1; CAD44798.1; NP_{—}055317.1; BAD96892.1; EAW89591.1; BAD96610.1; BAF94683.1; NP_{—}000190.1; EAW89590.1; AAI42657.1; EAW80552.1; NP_{—}002363.2; AAC50302.1; BAA10017.1; BAC04088.1; EAW95202.1; NP_{—}689668.3; Q8IWY8; NP_{—}758449.1; AAA60962.1; AA109272.1; AAH17179.2; EAW80549.1; BAC04147.1; EAW92602.1; EAW80551.1; EAW92601.1; AAI46792.1; EAX09103.1; CAD39107.2; BAC87143.1; BAC04608.1; BAA74888.2; NP_{—}055826.1; EAW80555.1; AAP46291.1; AF450110_{—}1; NP 758847.1; EAW81084.1; NP_{—}579875.1; AAK57543.1; EAW80550.1; EAW80554.1; AAA52898.1; EAW90131.1; EAW90130.1; EAW90129.1; EAX07172.1; EAW99553.1; EAW99554.1; BAF85296.1; BAC85557.1; NP_{—}001073944.1; EAW70428.1; EAW70429.1; CAC38371.1; AAA02487.1; CAA64990.1; NP_{—}003212.2; NP_{—}001014448.1; EAX04355.1; CAC38370.1; NP_{—}003211.1; EAX04357.1; NP_{—}001035890.1; NP_{—}001027451.1; CAA71047.1; AAB58911.1; NP_{—}003643.2; NP_{—}001014447.1; EAW55251.1; NP 758438.2; AAK16926.1; AAW65983.1; BAA86445.2; EAW82332.1; EAW65950.1; NP_{—}056197.2; AAP13073.1; Q9ULT8; AAH20812.1; Q6VUC0.1; AAF75765.1; AF265208_{—}1; NP 848643.1; EAW55076.1; NP_{—}056131.2; BAA34491.1; Q96KQ4; EAW81845.1; EAW81846.1; BAC86342.1; NP_{—}001073923.1; BAB71147.1; CAA12585.1; BAE06117.1; NP_{—}001099010.1; Q13574; EAW84195.1; NP_{—}056051.2; EAX05525.1; EAX05527.1; EAX05529.1; EAX05526.1; EAX05528.1; EAX05530.1; AAG27462.1; AF307080_{—}1; Q9HAR2; BAC86852.1; AAB39350.1; NP_{—}219482.1; BAA91375.1; EAW82665.1; EAW82664.1; EAW82663.1; AAH01249.2; Q5H8A4; NP_{—}060203.2; BAB55130.1; BAA91046.1; BAC11157.1; NP_{—}002185.1; XP_{—}941944.2; BAD92869.1; BAA31672.2; NP 942592.1; EAX05658.1; NP_{—}115593.3; Q92617; NP_{—}001073007.1; BAC87606.1; BAA13210.2; AAH43394.1; EAX05656.1; AAH07747.2; BAC05248.1; BAA91273.1; EAW50318.1; AAK68115.1; AAI07684.1; AAH04173.1; AAI19776.1; NP_{—}000732.2; 2OZB; NP_{—}060358.2; EAX10870.1; Q6NUN0.1; EAW50319.1; NP 478056.1; EAW93704.1; NP_{—}003213.1; AAH94882.1; EAW75331.1; AAH46933.1; NP_{—}006593.2; BAA36557.1; AAD53986.1; AF070992_{—}1; CAC24700.1; Q92665; NP_{—}005821.1; AAH22045.1; NP_{—}060212.3; BAB21835.1; CAD38618.1; AAA61276.1; Q9COB9.5; EAW92801.1; XP_{—}001131071.1; AAH26009.2; AAQ88673.1; AAH35645.1; AA027355.1; NP_{—}660333.2; Q81YS4; AAM22870.1; AF447587_{—}1; AAH35024.1; NP_{—}631909.2; EAW48495.1; Q6ZRQ5; NP_{—}940870.2; CAI16668.1; BAC87254.1; CAH18398.1; AAF61308.1; AF233442_{—}1; Q9UPN7.4; EAW61003.1; EAW61005.1; ABW05298.1; A5X5Y0.1; EAW78304.1; EAW60051.1; EAW60050.1; EAW83351.1; EAW68657.1; AA031694.1; AAH03130.2; CAB75649.1; EAW58527.1; EAW49556.1; BAA02656.1; NP_{—}001530.1; EAW82176.1; XP_{—}944430.2; XP_{—}291671.7; EAW52642.1; EAW80101.1; EAX10701.1; AAD28183.1; AF110334_{—}1; S34632; XP_{—}942788.2; EAX07176.1; Q8NBP7; AAV67948.1; NP_{—}777596.2; EAW97812.1; BAC92750.1; EAW90126.1; NP_{—}112598.1; BAC11572.1; EAW68655.1; NP_{—}110386.2; NP_{—}001014443.1; O015054; XP_{—}946876.1; NP_{—}001073893.1; BAA21572.2; AAH90946.1; EAW52645.1; CAB70895.1; AAG17222.1; AF217979_{—}1; CAH72142.1; EAW63132.1; EAW52643.1; P03971.2; AAC25614.1; AAA98805.1; NP_{—}000470.2; EAX08226.1; XP 498704.2; EAW69496.1; NP_{—}005874.1; AAA51898.1; NP_{—}000709.1; PH0268; BAC87589.1; EAX02325.1; EAW69497.1; BAD92136.1; XP_{—}001131894.1; EAX07870.1; EAW88415.1; NP_{—}543023.1; AAA52802.1; EAW69379.1; EAW69378.1; AAB00873.1; NP_{—}116126.2; A45023; EAX05944.1; BAD97263.1; NP_{—}116106.2; BAD38654.1; EAX11013.1; EAX11019.1; EAX11015.1; EAX11018.1; NP 597681.2; NP_{—}596869.3; NP_{—}597676.2; NP_{—}003310.3; Q8WZ42; CAD12455.1; CAD12456.1; CAA62188.1; AAH73136.1; DAA04877.1; NP_{—}001005241.1; NP_{—}001074310.1; EAW84621.1; BAC11307.1; NP_{—}078932.2; EAW84623.1; NP_{—}005413.2; EAW84659.1; EAW67518.1; EAW67517.1; AAH14170.2; AAH11917.2; AAC39742.2; AAD22671.1; AC0071922; O75443; NP_{—}005018.1; NP_{—}658988.2; EAW55645.1; EAW55644.1; AAL78337.1; AF467440_{—}1; AAI01118.1; EAW66773.1; EAW66767.1; EAW66766.1; EAW66769.1; EAW66772.1; EAW79650.1; NP_{—}001020244.1; NP_{—}001020243.1; AAH67743.1; NP_{—}056227.2; CAH18194.1; BAB14885.1; CAB55998.1; BAB15807.1; XP_{—}938889.2; DAA04758.1; NP_{—}001004723.1; ABJ97559.1; AAH25985.1; AAK51388.1; AAZ08682.1; CAE45505.1; BAC85373.1; BAC05019.1; AAR02476.1; AAA52865.1; CAD89406.1; EAW99256.1; EAW99258.1; EAW99259.1; EAW99257.1; CAA08737.1; AAZ08718.1; CAB44167.1; AAC18299.1; AAM87778.1; AF4604071; AAA59043.1; EAW94370.1; ABP98525.1; ABE66513.1; NP_{—}009066.1; Q15937; ABP98584.1; ABK80731.1; AAR02522.1; AAR38523.1; AAS86050.1; ABN58754.1; ABE67006.1; AAC18261.1; EAX04147.1; AAZ08800.1; CAK50686.1; AAR38715.1 and/or ABP98521.1.

In one embodiment, an isolated polypeptide of this invention may comprise a derivate of a polypeptide of this invention. “Derivative” is to be understood as referring, in some embodiments, to less than the full-length portion of the native sequence of the protein in question. In some embodiments, a “derivative” may further comprise (at its termini and/or within said sequence itself) non-native sequences, i.e. sequences which do not form part of the native protein in question. The term “derivative” also includes within its scope molecular species produced by conjugating chemical groups to the amino residue side chains of the native proteins or fragments thereof, wherein said chemical groups do not form part of the naturally-occurring amino acid residues present in said native proteins.

In one embodiment, the isolated polypeptide of this invention may include modification to the original sequence of the native protein. “Modification” is to be understood as comprising non-native amino acid residues and sequences of such non-native residues, which have been introduced as a consequence or mutation of the native sequence (by either random or site-directed processes).

In one embodiment, the polypeptide of this invention comprises an amino acid substitution. In one embodiment, the amino acid substitution is conservative. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In another embodiment, the amino acid substitution may not be conservative which may result in enhanced activity of the mutated polypeptide compared to the native polypeptide.

The polypeptides of this invention can be produced by any synthetic or recombinant process such as is well known in the art. Polypeptides can further be modified to alter biophysical or biological properties by means of techniques known in the art. For example, the polypeptide can be modified to increase its stability against proteases, or to modify its lipophilicity, solubility, or binding affinity to its native receptor.

Polypeptide homology for any polypeptide sequence listed herein may be determined by immunoblot analysis, or via computer algorithm analysis of amino acid sequences, utilizing any of a number of software packages available, via methods well known to one skilled in the art. Some of these packages may include the FASTA, BLAST, MPsrch or Scanps packages, and may employ the use of the Smith and Waterman algorithms, and/or global/local or BLOCKS alignments for analysis, for example.

Homology, as used herein, may refer to sequence identity, or may refer to structural identity, or functional identity. By using the term “homology” and other like forms, it is to be understood that any molecule, whether nucleic acid or peptide, that functions similarly, and/or contains sequence identity, and/or is conserved structurally so that it approximates the reference sequence, is to be considered as part of this invention.

The term “homology”, as used herein, indicates a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- or C-terminal extensions nor insertions shall be construed as reducing identity or homology. Methods and computer programs for the alignment are well known in the art.

In one embodiment, the terms “homology”, “homologue” or “homologous”, in any instance, indicate that the sequence referred to, whether an amino acid sequence, or a nucleic acid sequence, exhibits at least 70% correspondence with the indicated sequence. In another embodiment, the amino acid sequence or nucleic acid sequence exhibits at least 72% correspondence with the indicated sequence. In another embodiment, the amino acid sequence or nucleic acid sequence exhibits at least 75% correspondence with the indicated sequence. In another embodiment, the amino acid sequence or nucleic acid sequence exhibits at least 77% correspondence with the indicated sequence. In another embodiment, the amino acid sequence or nucleic acid sequence exhibits at least 80% correspondence with the indicated sequence. In another embodiment, the amino acid sequence or nucleic acid sequence exhibits at least 82% correspondence with the indicated sequence. In another embodiment, the amino acid sequence or nucleic acid sequence exhibits at least 85% correspondence with the indicated sequence. In another embodiment, the amino acid sequence or nucleic acid sequence exhibits at least 87% correspondence with the indicated sequence. In another embodiment, the amino acid sequence or nucleic acid sequence exhibits at least 90% correspondence with the indicated sequence. In another embodiment, the amino acid sequence or nucleic acid sequence exhibits at least 92% correspondence with the indicated sequence. In another embodiment, the amino acid sequence or nucleic acid sequence exhibits at least 95% or more correspondence with the indicated sequence. In another embodiment, the amino acid sequence or nucleic acid sequence exhibits 95%-100% correspondence to the indicated sequence. In another embodiment, the amino acid sequence or nucleic acid sequence exhibits 100% correspondence to the indicated sequence. Similarly, as used herein, the reference to a correspondence to a particular sequence includes both direct correspondence, as well as homology to that sequence as herein defined.

In one embodiment the polypeptide of this invention may be an isoform of the isolated polypeptide. In one embodiment, “isoform” refers to a version of a molecule, for example, a protein, with only slight differences to another isoform of the same protein. In one embodiment, isoforms may be produced from different but related genes, or in another embodiment, may arise from the same gene by alternative splicing. In another embodiment, isoforms are caused by single nucleotide polymorphisms.

In one embodiment the isolated polypeptide of this invention is a fragment of the native protein. In one embodiment, “fragment” refers to a protein or polypeptide that is shorter or comprises fewer amino acids than the full length protein or polypeptide. In another embodiment, fragment refers to a nucleic acid that is shorter or comprises fewer nucleotides than the full length nucleic acid. In another embodiment, the fragment is an N-terminal fragment. In another embodiment, the fragment is a C-terminal fragment. In one embodiment, the fragment of this invention is an intrasequential section of the protein, peptide, or nucleic acid. In another embodiment, the fragment is a functional intrasequential section of the protein, peptide or nucleic acid. In another embodiment, the fragment is a functional intrasequential section within the protein, peptide or nucleic acid. In another embodiment, the fragment is an N-terminal functional fragment. In one embodiment, the fragment is a C-terminal functional fragment. In another embodiment, the fragment is an N-terminal functional fragment. In another embodiment, the fragment is a C-terminal functional fragment. In one embodiment, a fragment has 10-20 nucleic or amino acids, while in another embodiment, a fragment has more than 5 nucleic or amino acids, while in another embodiment, a fragment has 100-200 nucleic or amino acids, while in another embodiment, a fragment has 100-500 nucleic or amino acids, while in another embodiment, a fragment has 50-200 nucleic or amino acids, while in another embodiment, a fragment has 10-250 nucleic or amino acids.

The term “polypeptide”, when in reference to any polypeptide of this invention, is meant to include native polypeptides (either degradation products, synthetically synthesized peptides or recombinant peptides) and peptidomimetics (typically, synthetically synthesized peptides), such as peptoids and semipeptoids which are peptide analogs, which may have, for example, modifications rendering the polypeptides more stable while in a body or more capable of penetrating into cells. Such modifications include, but are not limited to N terminal, C terminal or peptide bond modification, including, but not limited to, backbone modifications, and residue modification, each of which represents an additional embodiment of the invention. Methods for preparing peptidomimetic compounds are well known in the art and are specified, for example, in Quantitative Drug Design, C. A. Ramsden Gd., Chapter 17.2, F. Choplin Pergamon Press (1992). In one embodiment, a polypeptide is a full length protein or a variant of a known protein.

It is to be understood that any amino acid sequence, whether obtained naturally or synthetically by any means, exhibiting sequence, structural or functional homology to the polypeptides described herein, are considered part of this invention.

In some embodiments, this invention provides for antibodies specifically interacting with a polypeptide of this invention.

In some embodiments, the term “antibody” refers to intact molecules as well as functional fragments thereof, such as Fab, F(ab′)2, and Fv that are capable of specifcially interacting with a desired target as described herein, for example, binding to S. pneumoniae. In some embodiments, the antibody fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the variable region of the light chain and the variable region of the heavy chain expressed as two chains; and
(5) Single chain antibody (“SCA”), a genetically engineered molecule containing the variable region of the light chain and the variable region of the heavy chain, linked by a suitable polypeptide linker as a genetically fused single chain molecule.

Methods of making antibodies and antibody fragments are known in the art. (See for example, Harlow and Lane, Antibodies: A Laboratory Manual, Cold Spring Harbor Laboratory, New York, 1988, incorporated herein by reference).

In some embodiments, the antibody fragments may be prepared by proteolytic hydrolysis of the antibody or by expression in E. coli or mammalian cells (e.g. Chinese hamster ovary cell culture or other protein expression systems) of DNA encoding the fragment.

Antibody fragments can, in some embodiments, be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

In some embodiments, the antibodies or fragments as described herein may comprise “humanized forms” of antibodies. In some embodiments, the term “humanized forms of antibodies” refers to non-human (e.g. murine) antibodies, which are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al, Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g. mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The polypeptide utilized in methods and compositions of the present invention has, in one embodiment, the sequence SEQ ID No: 1. In another embodiment, a polypeptide utilized in methods and compositions of the present invention is a homologue of SEQ ID No: 1. In another embodiment, the polypeptide is an isoform of SEQ ID No: 1. In another embodiment, the protein is a variant of SEQ ID No: 1. In another embodiment, the protein is a fragment of SEQ ID No: 1. In another embodiment, the protein is a fragment of an isoform of SEQ ID No: 1. In another embodiment, the protein is a fragment of a variant of SEQ ID No: 1.

The polypeptide utilized in methods and compositions of the present invention has, in another embodiment, the sequence of SEQ ID No: 2. In another embodiment, a polypeptide utilized in methods and compositions of the present invention is a homologue of SEQ ID No: 2. In another embodiment, the polypeptide is an isoform of SEQ ID No: 2. In another embodiment, the protein is a variant of SEQ ID No: 2. In another embodiment, the protein is a fragment of SEQ ID No: 2. In another embodiment, the protein is a fragment of an isoform of SEQ ID No: 2. In another embodiment, the protein is a fragment of a variant of SEQ ID No: 2.

The polypeptide utilized in methods and compositions of the present invention has, in another embodiment, the sequence of SEQ ID No: 3. In another embodiment, a polypeptide utilized in methods and compositions of the present invention is a homologue of SEQ ID No: 3. In another embodiment, the polypeptide is an isoform of SEQ ID No: 3. In another embodiment, the protein is a variant of SEQ ID No: 3. In another embodiment, the protein is a fragment of SEQ ID No: 3. In another embodiment, the protein is a fragment of an isoform of SEQ ID No: 3. In another embodiment, the protein is a fragment of a variant of SEQ ID No: 3.

The polypeptide utilized in methods and compositions of the present invention has, in another embodiment, the sequence of SEQ ID No: 4. In another embodiment, a polypeptide utilized in methods and compositions of the present invention is a homologue of SEQ ID No: 4. In another embodiment, the polypeptide is an isoform of SEQ ID No: 4. In another embodiment, the protein is a variant of SEQ ID No: 4. In another embodiment, the protein is a fragment of SEQ ID No: 4. In another embodiment, the protein is a fragment of an isoform of SEQ ID No: 4. In another embodiment, the protein is a fragment of a variant of SEQ ID No: 4.

The polypeptide utilized in methods and compositions of the present invention has, in another embodiment, the sequence of SEQ ID No: 5. In another embodiment, a polypeptide utilized in methods and compositions of the present invention is a homologue of SEQ ID No: 5. In another embodiment, the polypeptide is an isoform of SEQ ID No: 5. In another embodiment, the protein is a variant of SEQ ID No: 5. In another embodiment, the protein is a fragment of SEQ ID No: 5. In another embodiment, the protein is a fragment of an isoform of SEQ ID No: 5. In another embodiment, the protein is a fragment of a variant of SEQ ID No: 5.

The polypeptide utilized in methods and compositions of the present invention has, in another embodiment, the sequence of SEQ ID No: 6. In another embodiment, a polypeptide utilized in methods and compositions of the present invention is a homologue of SEQ ID No: 6. In another embodiment, the polypeptide is an isoform of SEQ ID No: 6. In another embodiment, the protein is a variant of SEQ ID No: 6. In another embodiment, the protein is a fragment of SEQ ID No: 6. In another embodiment, the protein is a fragment of an isoform of SEQ ID No: 6. In another embodiment, the protein is a fragment of a variant of SEQ ID No: 6.

The polypeptide utilized in methods and compositions of the present invention has, in another embodiment, the sequence of SEQ ID No: 7. In another embodiment, a polypeptide utilized in methods and compositions of the present invention is a homologue of SEQ ID No: 7. In another embodiment, the polypeptide is an isoform of SEQ ID No: 7. In another embodiment, the protein is a variant of SEQ ID No: 7. In another embodiment, the protein is a fragment of SEQ ID No: 7. In another embodiment, the protein is a fragment of an isoform of SEQ ID No: 7. In another embodiment, the protein is a fragment of a variant of SEQ ID No: 7.

The polypeptide utilized in methods and compositions of the present invention has, in another embodiment, the sequence of SEQ ID No: 8. In another embodiment, a polypeptide utilized in methods and compositions of the present invention is a homologue of SEQ ID No: 8. In another embodiment, the polypeptide is an isoform of SEQ ID No: 8. In another embodiment, the protein is a variant of SEQ ID No: 8. In another embodiment, the protein is a fragment of SEQ ID No: 8. In another embodiment, the protein is a fragment of an isoform of SEQ ID No: 8. In another embodiment, the protein is a fragment of a variant of SEQ ID No: 8.

The polypeptide utilized in methods and compositions of the present invention has, in another embodiment, the sequence of SEQ ID No: 9. In another embodiment, a polypeptide utilized in methods and compositions of the present invention is a homologue of SEQ ID No: 9. In another embodiment, the polypeptide is an isoform of SEQ ID No: 9. In another embodiment, the protein is a variant of SEQ ID No: 9. In another embodiment, the protein is a fragment of SEQ ID No: 9. In another embodiment, the protein is a fragment of an isoform of SEQ ID No: 9. In another embodiment, the protein is a fragment of a variant of SEQ ID No: 9.

The polypeptide utilized in methods and compositions of the present invention has, in another embodiment, the sequence of SEQ ID No: 10. In another embodiment, a polypeptide utilized in methods and compositions of the present invention is a homologue of SEQ ID No: 10. In another embodiment, the polypeptide is an isoform of SEQ ID No: 10. In another embodiment, the protein is a variant of SEQ ID No: 10. In another embodiment, the protein is a fragment of SEQ ID No: 10. In another embodiment, the protein is a fragment of an isoform of SEQ ID No: 10. In another embodiment, the protein is a fragment of a variant of SEQ ID No: 10.

The polypeptide utilized in methods and compositions of the present invention has, in another embodiment, the sequence of SEQ ID No: 11. In another embodiment, a polypeptide utilized in methods and compositions of the present invention is a homologue of SEQ ID No: 11. In another embodiment, the polypeptide is an isoform of SEQ ID No: 11. In another embodiment, the protein is a variant of SEQ ID No: 11. In another embodiment, the protein is a fragment of SEQ ID No: 11. In another embodiment, the protein is a fragment of an isoform of SEQ ID No: 11. In another embodiment, the protein is a fragment of a variant of SEQ ID No: 11.

The polypeptide utilized in methods and compositions of the present invention has, in another embodiment, the sequence of SEQ ID No: 12. In another embodiment, a polypeptide utilized in methods and compositions of the present invention is a homologue of SEQ ID No: 12. In another embodiment, the polypeptide is an isoform of SEQ ID No: 12. In another embodiment, the protein is a variant of SEQ ID No: 12. In another embodiment, the protein is a fragment of SEQ ID No: 12. In another embodiment, the protein is a fragment of an isoform of SEQ ID No: 12. In another embodiment, the protein is a fragment of a variant of SEQ ID No: 12.

The polypeptide utilized in methods and compositions of the present invention has, in another embodiment, the sequence of SEQ ID No: 13. In another embodiment, a polypeptide utilized in methods and compositions of the present invention is a homologue of SEQ ID No: 13. In another embodiment, the polypeptide is an isoform of SEQ ID No: 13. In another embodiment, the protein is a variant of SEQ ID No: 13. In another embodiment, the protein is a fragment of SEQ ID No: 13. In another embodiment, the protein is a fragment of an isoform of SEQ ID No: 13. In another embodiment, the protein is a fragment of a variant of SEQ ID No: 13.

The polypeptide utilized in methods and compositions of the present invention has, in another embodiment, the sequence of SEQ ID No: 14. In another embodiment, a polypeptide utilized in methods and compositions of the present invention is a homologue of SEQ ID No: 14. In another embodiment, the polypeptide is an isoform of SEQ ID No: 14. In another embodiment, the protein is a variant of SEQ ID No: 14. In another embodiment, the protein is a fragment of SEQ ID No: 14. In another embodiment, the protein is a fragment of an isoform of SEQ ID No: 14. In another embodiment, the protein is a fragment of a variant of SEQ ID No: 14.

The polypeptide utilized in methods and compositions of the present invention has, in another embodiment, the sequence of SEQ ID No: 15. In another embodiment, a polypeptide utilized in methods and compositions of the present invention is a homologue of SEQ ID No: 15. In another embodiment, the polypeptide is an isoform of SEQ ID No: 15. In another embodiment, the protein is a variant of SEQ ID No: 15. In another embodiment, the protein is a fragment of SEQ ID No: 15. In another embodiment, the protein is a fragment of an isoform of SEQ ID No: 15. In another embodiment, the protein is a fragment of a variant of SEQ ID No: 15.

The polypeptide utilized in methods and compositions of the present invention has, in another embodiment, the sequence of SEQ ID No: 16. In another embodiment, a polypeptide utilized in methods and compositions of the present invention is a homologue of SEQ ID No: 16. In another embodiment, the polypeptide is an isoform of SEQ ID No: 16. In another embodiment, the protein is a variant of SEQ ID No: 16. In another embodiment, the protein is a fragment of SEQ ID No: 16. In another embodiment, the protein is a fragment of an isoform of SEQ ID No: 16. In another embodiment, the protein is a fragment of a variant of SEQ ID No: 16.

The polypeptide utilized in methods and compositions of the present invention has, in another embodiment, the sequence of SEQ ID No: 17. In another embodiment, a polypeptide utilized in methods and compositions of the present invention is a homologue of SEQ ID No: 17. In another embodiment, the polypeptide is an isoform of SEQ ID No: 17. In another embodiment, the protein is a variant of SEQ ID No: 17. In another embodiment, the protein is a fragment of SEQ ID No: 17. In another embodiment, the protein is a fragment of an isoform of SEQ ID No: 17. In another embodiment, the protein is a fragment of a variant of SEQ ID No: 17.

The polypeptide utilized in methods and compositions of the present invention has, in another embodiment, the sequence of SEQ ID No: 18. In another embodiment, a polypeptide utilized in methods and compositions of the present invention is a homologue of SEQ ID No: 18. In another embodiment, the polypeptide is an isoform of SEQ ID No: 18. In another embodiment, the protein is a variant of SEQ ID No: 18. In another embodiment, the protein is a fragment of SEQ ID No: 18. In another embodiment, the protein is a fragment of an isoform of SEQ ID No: 18. In another embodiment, the protein is a fragment of a variant of SEQ ID No: 18.

The polypeptide utilized in methods and compositions of the present invention has, in another embodiment, the sequence of SEQ ID No: 19. In another embodiment, a polypeptide utilized in methods and compositions of the present invention is a homologue of SEQ ID No: 19. In another embodiment, the polypeptide is an isoform of SEQ ID No: 19. In another embodiment, the protein is a variant of SEQ ID No: 19. In another embodiment, the protein is a fragment of SEQ ID No: 19. In another embodiment, the protein is a fragment of an isoform of SEQ ID No: 19. In another embodiment, the protein is a fragment of a variant of SEQ ID No: 19.

The polypeptide utilized in methods and compositions of the present invention has, in another embodiment, the sequence of SEQ ID No: 20. In another embodiment, a polypeptide utilized in methods and compositions of the present invention is a homologue of SEQ ID No: 20. In another embodiment, the polypeptide is an isoform of SEQ ID No: 20. In another embodiment, the protein is a variant of SEQ ID No: 20. In another embodiment, the protein is a fragment of SEQ ID No: 20. In another embodiment, the protein is a fragment of an isoform of SEQ ID No: 20. In another embodiment, the protein is a fragment of a variant of SEQ ID No: 20.

The polypeptide utilized in methods and compositions of the present invention has, in another embodiment, the sequence of SEQ ID No: 21. In another embodiment, a polypeptide utilized in methods and compositions of the present invention is a homologue of SEQ ID No: 21. In another embodiment, the polypeptide is an isoform of SEQ ID No: 21. In another embodiment, the protein is a variant of SEQ ID No: 21. In another embodiment, the protein is a fragment of SEQ ID No: 21. In another embodiment, the protein is a fragment of an isoform of SEQ ID No: 21. In another embodiment, the protein is a fragment of a variant of SEQ ID No: 21.

The polypeptide utilized in methods and compositions of the present invention has, in another embodiment, the sequence of SEQ ID No: 22. In another embodiment, a polypeptide utilized in methods and compositions of the present invention is a homologue of SEQ ID No: 22. In another embodiment, the polypeptide is an isoform of SEQ ID No: 22. In another embodiment, the protein is a variant of SEQ ID No: 22. In another embodiment, the protein is a fragment of SEQ ID No: 22. In another embodiment, the protein is a fragment of an isoform of SEQ ID No: 22. In another embodiment, the protein is a fragment of a variant of SEQ ID No: 22.

The polypeptide utilized in methods and compositions of the present invention has, in another embodiment, the sequence of SEQ ID No: 23. In another embodiment, a polypeptide utilized in methods and compositions of the present invention is a homologue of SEQ ID No: 23. In another embodiment, the polypeptide is an isoform of SEQ ID No: 23. In another embodiment, the protein is a variant of SEQ ID No: 23. In another embodiment, the protein is a fragment of SEQ ID No: 23. In another embodiment, the protein is a fragment of an isoform of SEQ ID No: 23. In another embodiment, the protein is a fragment of a variant of SEQ ID No: 23.

The polypeptide utilized in methods and compositions of the present invention has, in another embodiment, the sequence of SEQ ID No: 24. In another embodiment, a polypeptide utilized in methods and compositions of the present invention is a homologue of SEQ ID No: 24. In another embodiment, the polypeptide is an isoform of SEQ ID No: 24. In another embodiment, the protein is a variant of SEQ ID No: 24. In another embodiment, the protein is a fragment of SEQ ID No: 24. In another embodiment, the protein is a fragment of an isoform of SEQ ID No: 24. In another embodiment, the protein is a fragment of a variant of SEQ ID No: 24.

The polypeptide utilized in methods and compositions of the present invention has, in another embodiment, the sequence of SEQ ID No: 25. In another embodiment, a polypeptide utilized in methods and compositions of the present invention is a homologue of SEQ ID No: 25. In another embodiment, the polypeptide is an isoform of SEQ ID No: 25. In another embodiment, the protein is a variant of SEQ ID No: 25. In another embodiment, the protein is a fragment of SEQ ID No: 25. In another embodiment, the protein is a fragment of an isoform of SEQ ID No: 25. In another embodiment, the protein is a fragment of a variant of SEQ ID No: 25.

The polypeptide utilized in methods and compositions of the present invention has, in another embodiment, the sequence of SEQ ID No: 26. In another embodiment, a polypeptide utilized in methods and compositions of the present invention is a homologue of SEQ ID No: 26. In another embodiment, the polypeptide is an isoform of SEQ ID No: 26. In another embodiment, the protein is a variant of SEQ ID No: 26. In another embodiment, the protein is a fragment of SEQ ID No: 26. In another embodiment, the protein is a fragment of an isoform of SEQ ID No: 26. In another embodiment, the protein is a fragment of a variant of SEQ ID No: 26.

The polypeptide utilized in methods and compositions of the present invention has, in another embodiment, the sequence of SEQ ID No: 27. In another embodiment, a polypeptide utilized in methods and compositions of the present invention is a homologue of SEQ ID No: 27. In another embodiment, the polypeptide is an isoform of SEQ ID No: 27. In another embodiment, the protein is a variant of SEQ ID No: 27. In another embodiment, the protein is a fragment of SEQ ID No: 27. In another embodiment, the protein is a fragment of an isoform of SEQ ID No: 27. In another embodiment, the protein is a fragment of a variant of SEQ ID No: 27.

The polypeptide utilized in methods and compositions of the present invention has, in another embodiment, the sequence of SEQ ID No: 28. In another embodiment, a polypeptide utilized in methods and compositions of the present invention is a homologue of SEQ ID No: 28. In another embodiment, the polypeptide is an isoform of SEQ ID No: 28. In another embodiment, the protein is a variant of SEQ ID No: 28. In another embodiment, the protein is a fragment of SEQ ID No: 28. In another embodiment, the protein is a fragment of an isoform of SEQ ID No: 28. In another embodiment, the protein is a fragment of a variant of SEQ ID No: 28.

The polypeptide utilized in methods and compositions of the present invention has, in another embodiment, the sequence of SEQ ID No: 29. In another embodiment, a polypeptide utilized in methods and compositions of the present invention is a homologue of SEQ ID No: 29. In another embodiment, the polypeptide is an isoform of SEQ ID No: 29. In another embodiment, the protein is a variant of SEQ ID No: 29. In another embodiment, the protein is a fragment of SEQ ID No: 29. In another embodiment, the protein is a fragment of an isoform of SEQ ID No: 29. In another embodiment, the protein is a fragment of a variant of SEQ ID No: 29.

The polypeptide utilized in methods and compositions of the present invention has, in another embodiment, the sequence of SEQ ID No: 30. In another embodiment, a polypeptide utilized in methods and compositions of the present invention is a homologue of SEQ ID No: 30. In another embodiment, the polypeptide is an isoform of SEQ ID No: 30. In another embodiment, the protein is a variant of SEQ ID No: 30. In another embodiment, the protein is a fragment of SEQ ID No: 30. In another embodiment, the protein is a fragment of an isoform of SEQ ID No: 30. In another embodiment, the protein is a fragment of a variant of SEQ ID No: 30.

The polypeptide utilized in methods and compositions of the present invention has, in another embodiment, the sequence of SEQ ID No: 31. In another embodiment, a polypeptide utilized in methods and compositions of the present invention is a homologue of SEQ ID No: 31. In another embodiment, the polypeptide is an isoform of SEQ ID No: 31. In another embodiment, the protein is a variant of SEQ ID No: 31. In another embodiment, the protein is a fragment of SEQ ID No: 31. In another embodiment, the protein is a fragment of an isoform of SEQ ID No: 31. In another embodiment, the protein is a fragment of a variant of SEQ ID No: 31.

The polypeptide utilized in methods and compositions of the present invention has, in another embodiment, the sequence of SEQ ID No: 32. In another embodiment, a polypeptide utilized in methods and compositions of the present invention is a homologue of SEQ ID No: 32. In another embodiment, the polypeptide is an isoform of SEQ ID No: 32. In another embodiment, the protein is a variant of SEQ ID No: 32. In another embodiment, the protein is a fragment of SEQ ID No: 32. In another embodiment, the protein is a fragment of an isoform of SEQ ID No: 32. In another embodiment, the protein is a fragment of a variant of SEQ ID No: 32.

The polypeptide utilized in methods and compositions of the present invention has, in another embodiment, the sequence of SEQ ID No: 33. In another embodiment, a polypeptide utilized in methods and compositions of the present invention is a homologue of SEQ ID No: 33. In another embodiment, the polypeptide is an isoform of SEQ ID No: 33. In another embodiment, the protein is a variant of SEQ ID No: 33. In another embodiment, the protein is a fragment of SEQ ID No: 33. In another embodiment, the protein is a fragment of an isoform of SEQ ID No: 33. In another embodiment, the protein is a fragment of a variant of SEQ ID No: 33.

The polypeptide utilized in methods and compositions of the present invention has, in another embodiment, the sequence of SEQ ID No: 34. In another embodiment, a polypeptide utilized in methods and compositions of the present invention is a homologue of SEQ ID No: 34. In another embodiment, the polypeptide is an isoform of SEQ ID No: 34. In another embodiment, the protein is a variant of SEQ ID No: 34. In another embodiment, the protein is a fragment of SEQ ID No: 34. In another embodiment, the protein is a fragment of an isoform of SEQ ID No: 34. In another embodiment, the protein is a fragment of a variant of SEQ ID No: 34.

Nucleic acid sequences encoding for the above mentioned polypeptide amino acid consensus sequence comprise the present invention, as well. As used herein, the term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations as compared to wild type sequences, nevertheless encoding a protein or peptide, or fragments thereof, retaining wild type function exhibiting the same biological effect, despite these variations. Each of these represents a separate embodiment of this present.

The nucleic acids of the present invention can be produced by any synthetic or recombinant process such as is well known in the art. Nucleic acids according to the invention can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); Biotechnol Bioeng 1999 Oct. 5; 65(1):1-9 Prediction of antisense oligonucleotide binding affinity to a structured RNA target. Walton S P, Stephanopoulos G N, Yarmush M L, Roth C M.; Prediction of antisense oligonucleotide efficacy by in vitro methods. 0 Matveeva, B Felden, A Tsodikov, J Johnston, B P Monia, J F Atkins, R F Gesteland & S M Freier Nature Biotechnology 16, 1374-1375 (1998); all of which are incorporated by reference as if fully set forth herein. Other general references are provided throughout this document. The procedures therein are believed to be well known in the art and are provided for the convenience of the reader. All the information contained therein is incorporated herein by reference.

In another embodiment, nucleic acid sequences encoding the polypeptides amino acid are provided. For each of these it is to be understood that nucleic acid sequences encoding the peptides of this invention as described herein, are part of this invention and represent embodiments of this invention.

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein of interest. The invention further provides DNA sequences which encode proteins similar to those encoded by sequences as described herein, but which differ in terms of their codon sequence due to the degeneracy of the genetic code or allelic variations (naturally-occurring base changes in the species population which may or may not result in an amino acid change), which may encode the proteins of the invention described herein, as well. Variations in the DNA sequences, which are caused by point mutations or by induced modifications (including insertion, deletion, and substitution) to enhance the activity, half-life or production of the polypeptides encoded thereby, are also encompassed in the invention.

In another embodiment, this invention provides a vector comprising a nucleic acid sequence encoding polypeptides of the present invention.

The compositions of the present invention comprise an effective amount of one or more polypeptides as the active component, suspended in an appropriate vehicle. In the case of intranasal formulations, for example, said formulations may include vehicles that neither cause irritation to the nasal mucosa nor significantly disturb ciliary function. Diluents such as water, aqueous saline may also be added. The nasal formulations may also contain preservatives including, but not limited to, chlorobutanol and benzalkonium chloride. A surfactant may be present to enhance absorption of the subject proteins by the nasal mucosa. An additional mode of composition delivery may include an encapsulation technique, which involves complex coacervation of gelatin and chondroitin sulfate (Azhari R, Leong K. W. 1991. Complex coacervation of chondroitin sulfate and gelatin and its use for encapsulation and slow release of a model protein. Proc. Sympl. Control. Rel. 18:617; Brown K. E., Leong K., Huang C. H., Dalal R., Green G. D., Haimes H. B., Jimenez P. A., Bathon J. 1998. Gelatin/chondroitin 6-sulfate microspheres for delivery of therapeutic proteins to the joint. Arthritis Rheum. 41:2185-2195) all of which are incorporated by reference as if fully set forth herein.

Oral liquid preparations may be in the form of, for example, aqueous or oily suspension, solutions, emulsions, syrups or elixirs, or may be presented dry in table form or a product for reconstitution with water or other suitable vehicle before use. Such liquid preparations may contain conventional additives such as suspending agents, emulsifying agents, non-aqueous vehicles (which may include edible oils) or preservative.

Further examples of materials and methods useful in the preparation of polypeptide-containing compositions are well known to those skilled in the art. In addition, further details may be gleaned from Remington's Pharmaceutical Sciences, Mack Publishing Co., Easton, Pa., USA (1980) which is incorporated by reference as if fully set forth herein.

In one embodiment the compositions of this invention comprise a polypeptide of this invention, alone or in some embodiments, in combination with a second pharmaceutically active or therapeutic agent. In one embodiment, the term “pharmaceutically active agent” refers to any medicament which satisfies the indicated purpose. In some embodiments, the term “agent” of this invention is a decongestant, antibiotic, bronchodilator, anti-inflammatory steroid, leukotriene antagonist or histamine receptor antagonist, and the like.

In one embodiment, decongestants are medicines used to relieve nasal congestion caused by swelling of the membranes lining the nasal passages. Decongestants relieve the swelling by reducing the blood supply to the swollen membranes, causing the membranes to shrink. Although any suitable decongestant can be used, the preferred decongestants of the present invention are pseudoephedrine, a pharmaceutically acceptable pseudoephedrine salt, and mixtures thereof, as well as a phenylephrine salt. Pseudoephedrine is a sympathomimetic amine. Any suitable pseudoephedrine salt may be used in the present invention; however, pseudoephedrine hydrochloride, (+)-pseudoephedrine sulfate, and/or phenylephrine salt such as phenylephrine hydrochloride, are typically used. Other suitable pseudoephedrine salts include sodium, hydrofluoric, sulfuric, sulfonic, tartic, fumaric, hydrobromic, glycolic, citric, maleic, phosphoric, succinic, acetic, nitric, benzoic, ascorbic, p-toluene, benzenesulfonic, naphthalenesulfonic, propionic, and the like. In addition to pseudoephedrine, other suitable decongestants include oxymetazoline, phenylpropanolamine, and other sympathomimetic drugs.

In one embodiment, examples of leukotriene antagonist agents are montelukast and zafirlukast which block the actions of cysteinyl leukotrienes at the CysLT1 receptor on target cells such as bronchial smooth muscle.

In one embodiment, examples of bronchodilators are metaproterenol, isoetherine, terbutaline, albuterol and atropine sulfate.

In one embodiment, examples of histamine receptor antagonist are loratadine or desloratadine. Other antihistamines that may be utilized include H1 antagonist antihistamines including: ethylenediamines, such as mepyramine (pyrilamine) and antazoline; ethanolamines, such as diphenhydramine, carbinoxamine, doxylamine, clemastine, dimenhydrinate; alkylamines, such as pheniramine, chlorphenamine (chlorpheniramine), dexchlorphenamine, brompheniramine, triprolidine; piperazines, such as hydroxyzine and meclizine; tricyclics, such as promethazine, alimemazine (trimeprazine), cyproheptadine, azatadine; acrivastine; astemizole; cetirizine, levocetirizine, fexofenadine, loratadine, desloratadine, mizolastine, and terfenadine.

In one embodiment, the medicament is an anti-infective agent. In one embodiment, the anti-infective agent is an antibiotic agent. In one embodiment, the antibiotic agent is a beta-lactam antibiotic. In one embodiment, beta-lactam antibiotics include, but are not limited to, penicillin, benzathine penicillin, benzylpenicillin, amoxicillin, procaine penicillin, dicloxacillin, amoxicillin, flucloxacillin, ampicillin, methicillin, azlocillin, carbenicillin, ticarcillin, mezlocillin, piperacillin, phenoxymethylpenicillin, co-amoxiclav, cephalosporin, cefalexin, cephalothin, cefazolin, cefaclor, cefuroxime, cefamandole, cefotetan, cefoxitin, ceftriaxone, cefotaxime, ceftazidime, cefepime, cefpirome, imipenem, meropenem, ertapenem, faropenem, monobactam, aztreonam, or carbapenem.

In one embodiment, the antibiotic is a tetracycline antibiotic. In one embodiment, tetracycline antibiotics include, but are not limited to, tetracycline, chlortetracycline, demeclocycline, doxycycline, lymecycline, minocycline, or oxytetracycline.

In one embodiment, the antibiotic is a macrolide antibiotic. In one embodiment, macrolide antibiotics include, but are not limited to, erythromycin, azithromycin, oxithromycin, dirithromycin, clarithromycin, josamycin, oleandomycin, kitasamycin, spiramycin, tylosin/tylocine, troleandomycin, carbomycin, cethromycin, or telithromycin.

In one embodiment, the antibiotic is an aminoglycoside antibiotic. In one embodiment, aminoglycoside antibiotics include, but are not limited to, gentamicin, tobramycin, faropenem, imipenem, kanamycin, neomycin, ertapenem, apramycin, paromomycin sulfate, streptomycin, or amikacin.

In one embodiment, the antibiotic is a quinolone antibiotic. In one embodiment, quinolone antibiotics include, but are not limited to, ciprofloxacin, norfloxacin, lomefloxacin, enoxacin, ofloxacin, ciprofloxacin, levofloxacin, sparfloxacin, gatifloxacin, moxifloxacin, trovafloxacin, or alatrofloxacin.

In one embodiment, the antibiotic is a cyclic peptide antibiotic. In one embodiment, cyclic peptide antibiotics include, but are not limited to, vancomycin, streptogramins, Microcin J25, Bacteriocin AS-48, RTD-1, or polymyxins.

In one embodiment, the antibiotic is a lincosamide antibiotic. In one embodiment, lincosamide antibiotics include, but are not limited to, clindamycin.

In one embodiment, the antibiotic is an oxazolidinone antibiotic. In one embodiment, oxazolidinone antibiotics include, but are not limited to, linezolid, U-100592, DA-7867, AZD2563, or U-100766.

In one embodiment, the antibiotic is a sulfa antibiotic. In one embodiment, sulfa antibiotics include, but are not limited to, sulfisoxazole.

In one embodiment, the antibiotic is an antiseptic agent. In one embodiment, antiseptic agents include, but are not limited to, alcohols, chlorhexidine, chlorine, hexachlorophene, iodophors, chloroxylenol (PCMX), quaternary ammonium compounds, or triclosan.

In one embodiment, the medicament may be a growth factor such as epidermal growth factor (EGF), transforming growth factor-α(TGF-α), platelet derived growth factor (PDGF), fibroblast growth factors (FGFs) including acidic fibroblast growth factor (α-FGF) and basic fibroblast growth factor (β-FGF), transforming growth factor-β (TGF-β) and insulin like growth factors (IGF-1 and IGF-2), or any combination thereof.

In one embodiment, the medicament may be a local anesthetic agent. In one embodiment, local anesthetic agents include, but are not limited to benzocaine, chloroprocaine, cocaine, procaine, bupivacaine, levobupivacaine, lidocaine, mepivacaine, prilocalne, or ropivacaine. In one embodiment, the medicament may be a general anaesthetic agent. In one embodiment, general anesthetic agents include, but are not limited to, esflurane, sevoflurane, isoflurane, halothane, enflurane, methoxyflurane, xenon, propofol, etomidate, methohexital, midazolam, diazepamor, ketamine, thiopentone/thiopental, or lidocaine/prilocalne.

In one embodiment, the medicament may be an analgesic agent. In some embodiments, analgesic agents include, but are not limited to, paracetamol or non-steroidal anti-inflammatory agent. In some embodiments, analgesic agents include opiates or morphinomimetics such as morphine, pethidine, oxycodone, hydrocodone, diamorphine, tramadol, or buprenorphine. In some embodiments, a combination of two or more analgesics is desired.

In one embodiment, the medicament may be a sedative agent. In one embodiment, the sedative agent is an antidepressant agent such as mirtazapine or trazodone. In one embodiment, the sedative agent is a barbiturate such as secobarbital, pentobarbital, or amobarbital. In one embodiment, the sedative agent is a benzodiazepine such as diazepam, clonazepam, alprazolam, temazepam, chlordiazepoxide, flunitrazepam, lorazepam, or clorazepate. In one embodiment, the sedative agent is an imidazopyridines such as zolpidem or alpidem. In one embodiment, the sedative agent is a pyrazolopyrimidine such as zaleplon. In one embodiment, the sedative agent is an antihistamine such as diphenhydramine, dimenhydrinate, or doxylamine. In one embodiment, the sedative agent is an antipsychotic agent such as ziprasidone, risperidone, quetiapine, clozapine, prochlorperazine, perphenazine, loxapine, trifluoperazine, thiothixene, haloperidol, or fluphenazine. In one embodiment, the sedative agent is an herbal sedative such as valerian plant mandrake, or kava. In some embodiments, the sedative agent is eszopiclone, ramelteon, methaqualone, ethchlorvynol, chloral hydrate, meprobamate, glutethimide, methyprylon, gamma-hydroxybutyrate, ethyl alcohol, methyl trichloride, zopiclone, or diethyl ether.

In one embodiment, the medicament is an agent for treating a wasting disease. In some embodiments, agents treating a wasting disease include, but are not limited to, corticosteroids, anabolic steroids, cannabinoids, metoclopramid, cisapride, medroxyprogesterone acetate, megestrol acetate, cyproheptadine, hydrazine sulfate, pentoxifylline, thalidomide, anticytokine antibodies, cytokine inhibitors, eicosapentaenoic acid, indomethacin, ibuprofen, melatonin, insulin, growth hormone, clenbuterol, porcine pancreas extract, IGF-1, IGF-1 analogue and secretagogue, myostatin analogue, proteasome inhibitor, testosterone, oxandrolone, enbrel, melanocortin 4 receptor agonist, or a combination thereof.

In one embodiment, the medicaments are anti-inflammatory agents. In one embodiment, the anti-inflammatory agent is a non-steroidal anti-inflammatory agent. In one embodiment, the non-steroidal anti-inflammatory agent is a cox-1 inhibitor. In one embodiment, the non-steroidal anti-inflammatory agent is a cox-2 inhibitor. In one embodiment, the non-steroidal anti-inflammatory agent is a cox-1 and cox-2 inhibitor. In some embodiments, non-steroidal anti-inflammatory agents include but are not limited to aspirin, salsalate, diflunisal, ibuprofen, fenoprofen, flubiprofen, fenamate, ketoprofen, nabumetone, piroxicam, naproxen, diclofenac, indomethacin, sulindac, tolmetin, etodolac, ketorolac, oxaprozin, or celecoxib. In one embodiment, the anti-inflammatory agent is a steroidal anti-inflammatory agent. In one embodiment, the steroidal anti-inflammatory agent is a corticosteroid.

In one embodiment, the route of administration may be parenteral, or a combination thereof. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation (aerosol), nasal aspiration (spray), intranasal (drops), sublingual, oral, aerosol or suppository or a combination thereof. In one embodiment, the dosage regimen will be determined by skilled clinicians, based on factors such as exact nature of the condition being treated, the severity of the condition, the age and general physical condition of the patient, body weight, and response of the individual patient, etc.

For intranasal administration or application by inhalation, solutions or suspensions of the compounds mixed and aerosolized or nebulized in the presence of the appropriate carrier suitable. Such an aerosol may comprise any agent described herein.

For parenteral application, particularly suitable are injectable, sterile solutions, preferably oily or aqueous solutions, as well as suspensions, emulsions, or implants, including suppositories and enemas. Ampoules are convenient unit dosages. Such a suppository may comprise any agent described herein.

Sustained or directed release compositions can be formulated, e.g., liposomes or those wherein the active compound is protected with differentially degradable coatings, e.g., by microencapsulation, multiple coatings, etc. Such compositions may be formulated for immediate or slow release. It is also possible to freeze-dry the new compounds and use the lyophilisates obtained, for example, for the preparation of products for injection.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

In one embodiment, compositions of this invention are pharmaceutically acceptable. In one embodiment, the term “pharmaceutically acceptable” refers to any formulation which is safe, and provides the appropriate delivery for the desired route of administration of an effective amount of at least one compound for use in the present invention. This term refers to the use of buffered formulations as well, wherein the pH is maintained at a particular desired value, ranging from pH 4.0 to pH 9.0, in accordance with the stability of the compounds and route of administration.

In one embodiment, a composition of or used in the methods of this invention may be administered alone or within a composition. In another embodiment, compositions of this invention admixture with conventional excipients, i.e., pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. In another embodiment, the pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

In one embodiment, polypeptides of the present invention are administered in combination with a vaccine. In some embodiments, the vaccine is directed against S. pneumoniae, preventing the development of infection. In one embodiment, treatment or therapeutic vaccines are administered to patients and are designed to strengthen the body's natural defenses against S. pneumoniae infection. In one embodiment, therapeutic vaccines may prevent additional infections of S. pneumoniae. In some embodiments, prevention or prophylactic vaccines are administered to healthy individuals and are designed for individuals who present high risk for the disease.

In some embodiments, administration of the compounds of this invention is intended to reduce the severity of the pathologic condition. By the term “reduce the severity of the pathologic condition”, it is to be understood that any reduction via the methods, compounds and compositions disclosed herein, is to be considered encompassed by the invention. Reduction in severity may, in one embodiment comprise enhancement of survival, or in another embodiment, halting disease progression, or in another embodiment, delay in disease progression.

In some embodiments, administration of the compounds of this invention is intended to prevent or treat sepsis associated with S. pneumoniae infection. In one embodiment, treatments of this invention include the administration of a polypeptide, vector, nucleic acid, composition or therapeutic vaccines as herein described, administered to patients, whereby administration reduces the incidence, severity, or symptomatology associated with sepsis. In one embodiment, treatment of sepsis or latter stages of infection in a subject may be accomplished by the administration of any polypeptide of this invention, as herein described.

Dosing is dependent on the cellular responsiveness to the administered molecules/compounds or compositions comprising same. In general, the doses utilized for the above described purposes will vary, but will be in an effective amount to exert the desired effect, as determined by a clinician of skill in the art. As used herein, the term “pharmaceutically effective amount” refers to an amount of a compound as described herein, which will produce the desired alleviation in symptoms or other desired phenotype in a patient.

In one embodiment of the invention, the concentrations of the compounds will depend on various factors, including the nature of the condition to be treated, the condition of the patient, the route of administration and the individual tolerability of the compositions.

In some embodiments, any of the compositions of this invention will comprise a compound, in any form or embodiment as described herein. In some embodiments, any of the compositions of this invention will consist of a compound, in any form or embodiment as described herein. In some embodiments, any of the compositions of this invention will consist essentially of a compound, in any form or embodiment as described herein. In some embodiments, the term “comprise” refers to the inclusion of the indicated active agent, such as the compound of this invention, as well as inclusion of other active agents, and pharmaceutically acceptable carriers, excipients, emollients, stabilizers, etc., as are known in the pharmaceutical industry.

In some embodiments, the compositions of this invention will consist essentially of a polypeptide/polynucleotide/vector as herein described. In some embodiments, the term “consisting essentially of” refers to a composition whose only active ingredient of a particular class of agents, is the indicated active ingredient, however, other compounds may be included which are involved directly in the therapeutic effect of the indicated active ingredient. In some embodiments, the term “consisting essentially of” refers to a composition whose only active ingredient of targeting a particular mechanism, or acting via a particular pathway, is the indicated active ingredient, however, other compounds may be included which are involved directly in the therapeutic effect of the indicated active ingredient, which for example have a mechanism of action related to but not directly to that of the indicated agent. In some embodiments, the term “consisting essentially of” refers to a composition whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

It will be appreciated that the actual preferred amounts of active compound in a specific case will vary according to the specific compound being utilized, the particular compositions formulated, the mode of application, and the particular conditions and organism being treated. Dosages for a given host can be determined using conventional considerations, e.g., by customary comparison of the differential activities of the subject compounds and of a known agent, e.g., by means of an appropriate, conventional pharmacological protocol.

In one embodiment, the compounds of the invention may be administered acutely for acute treatment of temporary conditions, or may be administered chronically, especially in the case of progressive, recurrent, or degenerative disease. In one embodiment, one or more compounds of the invention may be administered simultaneously, or in another embodiment, they may be administered in a staggered fashion. In one embodiment, the staggered fashion may be dictated by the stage or phase of the disease.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions of this invention may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., aspartame, citric acid), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Furthermore, in another embodiment, the pharmaceutical compositions of this invention are administered as a suppository, for example a rectal suppository or a urethral suppository. Further, in another embodiment, the pharmaceutical compositions are administered by subcutaneous implantation of a pellet. In a further embodiment, the pellet provides for controlled release of an agent over a period of time. In yet another embodiment, the pharmaceutical compositions are administered in the form of a capsule.

In one embodiment, the pharmaceutical compositions provided herein are controlled-release compositions, i.e. compositions in which the anti-estrogen compound is released over a period of time after administration. Controlled- or sustained-release compositions include formulation in lipophilic depots (e.g., fatty acids, waxes, oils). In another embodiment, the composition is an immediate-release composition, i.e. a composition in which all of the compound is released immediately after administration. In one embodiment, the controlled- or sustained-release compositions of the invention are administered as a single dose. In another embodiment, compositions of the invention are administered as multiple doses, over a varying time period of minutes, hours, days, weeks, months or more. In another embodiment, compositions of the invention are administered during periods of acute disease. In another embodiment, compositions of the invention are administered during periods of chronic disease. In another embodiment, compositions of the invention are administered during periods of remission. In another embodiment, compositions of the invention are administered prior to development of gross symptoms.

In yet another embodiment, the pharmaceutical composition of this invention can be delivered in a controlled release system. For example, the agent may be administered using intravenous infusion, an implantable osmotic pump, a transdermal patch, liposomes, or other modes of administration. In one embodiment, a pump may be used. In another embodiment, polymeric materials can be used. In yet another embodiment, a controlled release system can be placed in proximity to the therapeutic target, i.e., the brain, thus requiring only a fraction of the systemic dose. In another embodiment, the controlled-release system may be any controlled release system known in the art.

The compositions may also include incorporation of the active material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

The preparation of pharmaceutical compositions that contain an active component, for example by mixing, granulating, or tablet-forming processes, is well understood in the art. The active therapeutic ingredient is often mixed with excipients that are pharmaceutically acceptable and compatible with the active ingredient. For oral administration, the compound is mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the compound is converted into a solution, suspension, or emulsion, if desired with the substances customary and suitable for this purpose, for example, solubilizers or other substances.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts are pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts, which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

It is the aim of the method of this invention to treat, inhibit, reduce or abrogate S. pneumoniae infection in subjects by administering polypeptides that inhibit the adhesion of S. pneumoniae to the respiratory tract.

As used herein, the term “treating” includes preventive as well as disorder remittive treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

As used herein, the term “administering” refers to bringing a subject in contact with a compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

Although the pharmaceutical compositions provided herein are principally directed to pharmaceutical compositions which are suitable for administration to humans, it will be understood by the skilled artisan that such compositions are generally suitable for administration to animals of all sorts. Modification of pharmaceutical composition suitable for administration to humans in order to render the compositions suitable for administration to various animals is well understood, and the ordinarily skilled veterinary pharmacologist can design and perform such modification with little, if any, experimentation. Subjects to which administration of the pharmaceutical compositions of the invention is contemplated include, but are not limited to, humans and other primates, and other mammals.

In one embodiment, “preventing, or treating” refers to any one or more of the following: delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics. In one embodiment, “treating” refers to both therapeutic treatment and prophylactic or preventive measures, wherein the object is to prevent or lessen the targeted pathologic condition or disorder as described hereinabove.

In another embodiment, “symptoms” may be any manifestation of a disease or pathological condition as described hereinabove.

The administration mode of the compounds and compositions of the present invention, timing of administration and dosage, i.e. the treatment regimen, will depend on the type and severity of the disease and the age and condition of the subject. In one embodiment, the compounds and compositions may be administered concomitantly. In another embodiment, the compounds and compositions may be administered at time intervals of seconds, minutes, hours, days, weeks or more.

In one embodiment, the treatment methods and composition of this invention are aimed at humans who are susceptible to S. pneumoniae infection. In one embodiment “a human subject susceptible”, is a member of a population who is at risk of becoming infected by a disease, if he or she is exposed to the infectious agent. In one embodiment, susceptibility to infection may result from a human being immune compromised due to other diseases or conditions such as AIDS or other immune system diseases. In another embodiment, a state of immune compromised may be imposed on a subject as a course of treatment, for example cancer chemotherapy or organ transplant. In another embodiment, the subject may suffer from respiratory diseases. These include diseases of the lung, pleural cavity, bronchial tubes, trachea, upper respiratory tract and of the nerves and muscles of breathing. Respiratory disease ranges from mild and self-limiting such as the common cold to life-threatening such as bacterial pneumonia or pulmonary embolism. Examples of obstructive respiratory diseases include, but are not limited to, chronic obstructive pulmonary disease, cystic fibrosis, asthma, bronchospasm, bronchitis, bronchiolitis, cronchiectasis, allergic bronchopulmonary aspergillosis, lung scarring after tuberculosis infection and pneumonia. Examples of restrictive lung diseases include, but are not limited to, asbestosis, radiation fibrosis, certain drugs such as amiodarone, bleomycin and methotrexate, rheumatoid arthritis, hypersensitivity pneumonitis, acute respiratory distress syndrome and infant respiratory distress syndrome. Other respiratory diseases are grouped as respiratory tract infections which include common cold, sinusitis, tonsillitis, otitis media, pharyngitis and laryngitis.

In one embodiment, the subject for treatment is a mammal. In another embodiment the subject is human. In another embodiment, the subject is defined as elderly human. In one embodiment the definition of “elderly human” is a person over 65 years of age. In another embodiment, “elderly” is defined by immune-compromised due to thymic involution. In one embodiment, the subject for treatment is a child, whereby the definition of a “child” is a human under 4 years of age. In another embodiment, a “child” refers to a human who is immune naïve, i.e. does not possess a fully developed immune system.

In one embodiment, the compositions described herein are aimed at treating or preventing sepsis caused by S. pneumonia infection in an individual. “Sepsis” is defined by the presence of bacteria (bacteremia) or other infectious organisms or their toxins in the blood (septicemia) or in other tissue of the body. Sepsis may be associated with clinical symptoms of systemic illness, such as fever, chills, malaise, low blood pressure, and mental status changes.

It is to be understood that reference to any publication, patent application or issued patent is to be considered as fully incorporated herein by reference in its entirety.

It is to be understood that this invention provides compositions, kits and uses of any combination of any agents as described herein, and such combinations represent embodiments of this invention.

It is to be understood that any method of this invention, as herein described, encompasses the administration of a compound as herein described, or a composition comprising the same, to the subject, in order to treat the indicated disease, disorder or condition. The methods as herein described each and/or all may further comprise administration of an additional therapeutic agent as herein described, and as will be appreciated by one skilled in the art.

It is to be understood that any assay for measuring a particular activity which is modulated by the therapeutic compound may be employed, as a means of determining the efficacy of the compound, in one embodiment, optimal loading of the compound, in another embodiment, timing and dosage, in another embodiment, or a combination thereof.

Any number of cells or cell lines may be incubated with tagged molecules and targeting of desired cells and/or uptake may be demonstrated by conventional means, including microscopy, FACS analysis, western blot analysis, and others.

Imaging methods include in vivo imaging, MR-imaging or NIRF analysis, as well as fluorescence microscopy of excised target tissue, the images of which may be compared to those obtained by MIR or NIRF.

The following examples are intended to illustrate but not limit the present invention.

### EXAMPLES

### Materials

Bacterial strains used in the following examples are shown below in Table 2.

TABLE 2

| | |
|---|---|
| Strain | Capsule type |

(a) S. pneumoniae capsulated types

| | |
|---|---|
| D39 | 2 |
| WU2 | 3 |
| 6BR | 6B |
| 9VR | 9V |
| 14DW | 14 |
| 14R | 14 |

(b) S. pneumoniae unencapsulated types

| | |
|---|---|
| R6 | 2 |
| 3.8 | 3 |
| 14.8 | 14 |

E. coli

DH5α UltraMAX
BL21(DE3)pLysS

The parental strains for the unencapsulated strains R6, 3.8 and 14.8 are D39, WU2 and 14DW, respectively.

### Results

### Example 1

### Identification of Phages which Bind to GtS

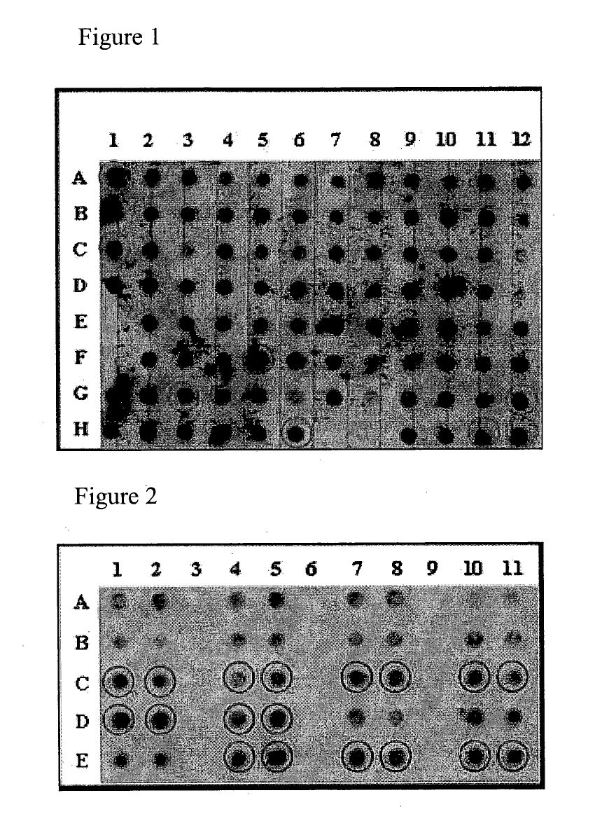

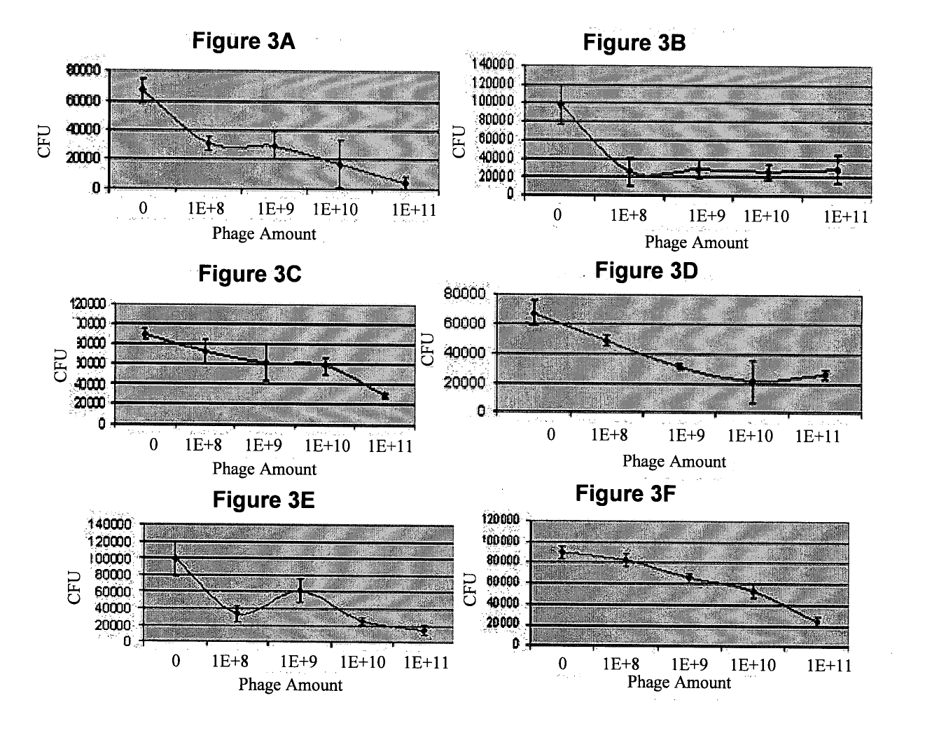

The filamentous bacteriophage fd, which carries a combinatorial random phage library inserted into phage envelope proteins (Enshell-Seijffers D et al., Nucleic Acids Res. 2001 29(10):E50-0), was utilized to identify phage, which bind to GtS. FIG. 1 shows nitrocellulose paper to which GtS protein had been bound, followed by incubation with the phage library. Circled spots indicate phages exhibiting the strongest binding. Confirmation of results was obtained by incubating nitrocellulose paper to which rGtS protein had been bound with specific anti-rGtS antibodies and anti-rabbit IgG (FIG. 2). Circled spots indicate the strongest phage binding identified by anti-rGtS antibodies.

### Example 2

### Inhibition of Adhesion of S. pneumoniae to Lung Epithelial Cells by Phages

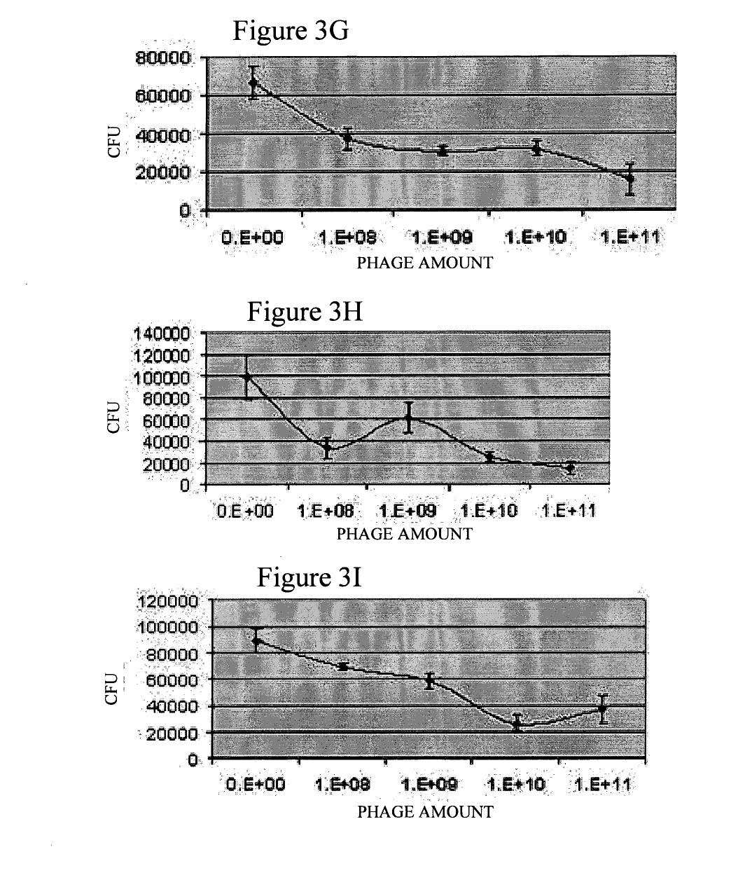

The phages that most strongly bound GtS were tested for their ability to inhibit adhesion of S. pneumoniae bacteria (serotype 3 strain WU2) to the A549 lung epithelial cell line. Nine phages were selected on the basis of having the strongest binding to the protein. All nine selected phages inhibited adhesion of the bacteria to the epithelial cells (FIG. 3A-I) FIG. 3A shows inhibition of adhesion by phage G12. FIG. 3B shows inhibition of adhesion by phage E9. FIG. 3C shows inhibition of adhesion by phage F5. FIG. 3D shows inhibition of adhesion by phage H12. FIG. 3E shows inhibition of adhesion by phage H11. FIG. 3F shows inhibition of adhesion by phage G3. FIG. 3G shows inhibition of adhesion by phage A1. FIG. 3H shows inhibition of adhesion by phage H11. FIG. 3I shows inhibition of adhesion by phage H6. Increased phage concentration correlated with a reduction in the binding ability of the bacteria to the epithelial cells. Inhibition of adhesion was significant and correlated with the number of phages in the culture.

### Example 3

### Sequences of Phages which Inhibit Adhesion of S. pneumoniae to Lung Epithelial Cells

In order to identify the peptide sequences which caused inhibition of adhesion, DNA sequences of the positive phages were prepared by the mini-prep method. DNA fragments were sequenced and particular sequences were chosen. Table 3 shows the nucleotide sequences from phage identified as inhibiting adhesion to the greatest degree.

TABLE 3
Nucleotide Sequences from Phage Showing
Greatest Adhesion Inhibition.

| SEQ ID NOs | Nucleotide Sequence |
|---|---|
| 35 | TCTCAGATTACTTTACGCTTAGTCTCTCGC |
| 36 | CATCGCTAGCATGAAAATTCTAAACAGAAAGGTGAA |
| 37 | GGTAAATCTCAGGATGGTTAGCGCCCTTGGCCTGAA |
| 38 | GCGAGGTAGGATGCCTAGGCTTGTAAGAATGTGAAT |
| 39 | GAGGGGCCTGCGAATTAGACGCAGAAGCATTCGATG |
| 40 | CGTTCGGGTCGTGATAATCAGTATTGGTAGATGACG |
| 41 | GCTTTACGCCGCAAAGCTGCTAATGTCGCTCGCACT |
| | TCTGCTGAACAG |
| 42 | GGGCCGTAGGGGGGGAGGGCGCAGAGGGAGGCGAAG |
| 43 | TATGCTATGCCTATTTAGCAGACGAAGTTTTAGTAT |

### Example 4

### Search for Human Homolog Protein as a Possible Candidate for the Target Protein

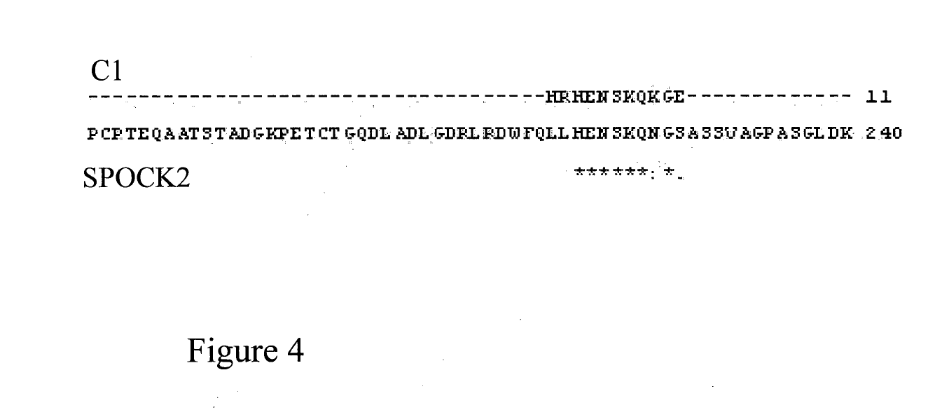

A search of the NCBI protein BLAST database was carried out using the BLASTp algorithm, probing the database for non-redundant protein sequences limited to mammalian or human datasets. The peptide sequences from positive phages were probed against the human genome. The peptides identified, Table 1 SEQ ID NOs: 10-34, comprise polypeptides of the invention. The peptides identified were selected from an analysis of a number of analogous proteins. From an analysis of the identified sequences, the human protein SPOCK2, was identified as exhibiting a homologous region (87% homology) to the C1 phage. FIG. 4 shows the local alignment between C1 and the homologous region in the SPOCK2 sequence. (*) perfect alignment, (:) conservative change and (.) semi-conservative change. Other protein sequences exhibiting homologous regions to the identified peptide sequences described herein, are described in the body of this specification and in the claims.

1.-51. (canceled)

52. An isolated polypeptide, wherein said polypeptide comprises an amino acid sequence at least 95% homologous to that set forth in SEQ ID NOs: 10, 11, 12, 13, 14, 15, 16, 17, 18 or 19, 1-9, or 20-34.

53. The isolated polypeptide of claim 52, wherein said polypeptide comprises:
a human laminin alpha 5 polypeptide consisting of an amino acid sequence as set forth in SEQ ID NO 10;
a nephronectin polypeptide consisting of an amino acid sequence as set forth in SEQ ID NO 11-12;
a SPOCK2 polypeptide consisting of an amino acid sequence as set forth in SEQ ID NOs 13-14;
a SILV silver homolog polypeptide consisting of an amino acid sequence as set forth in SEQ ID NOs 15-17;
a SPARC-like 1 polypeptide consisting of an amino acid sequence as set forth in SEQ ID NOs 18-19;
an insulin-like growth factor 2 (somatomedin A) polypeptide consisting of an amino acid sequence as set forth in SEQ ID NO 20;
a cadherin polypeptide consisting of an amino acid sequence as set forth in SEQ ID NOs 21-22;
a desmocollin 2 precursor polypeptide consisting of an amino acid sequence as set forth in SEQ ID NOs 23-24;
a tectorin alpha precursor polypeptide consisting of an amino acid sequence as set forth in SEQ ID NOs 25-26;
a FRAS1 related extracellular matrix 1 polypeptide consisting of an amino acid sequence as set forth in SEQ ID NO 27;
a metalloproteinase or metalloelastase polypeptide consisting of an amino acid sequence as set forth in SEQ ID NO 28;
a carboxypeptidase Z polypeptide consisting of an amino acid sequence as set forth in SEQ ID NOs 29-30;
a lactrophilin 3 polypeptide consisting of an amino acid sequence as set forth in SEQ ID NOs 31-32; or
a glycosyltransferase 25 domain polypeptide consisting of an amino acid sequence as set forth in SEQ ID NOs 33-34.

54. A composition comprising the isolated polypeptide of claim 52.

55. The composition of claim 54, wherein said composition further comprises one or more pharmaceutically active agents as decongestants, antibiotics, bronchodilators, anti-inflammatory steroids, leukotriene antagonists or histamine receptor antagonists, or a combination thereof.

56. The composition according to claim 54, wherein said composition is formulated for administration systemically, locally, by nasal inhalation, topically or a combination thereof.

57. A method of treating or preventing S. pneumoniae infection in a subject, said method comprising administering to said subject a polypeptide of claim 52, in an amount sufficient to inhibit or abrogate S. pneumoniae infection.

58. The method according claim 57, wherein the subject is a human subject and said subject is susceptible to S. pneumoniae infection, said subject is susceptible to, or is suffering from asthma, bronchospasm, bronchitis, bronchiolitis, and pneumonia, said subject is elderly or said subject is a child.

59. The method according to claim 57, wherein said human subject is further administered a decongestant, antibiotics, bronchodilators, anti-inflammatory steroids, leukotriene antagonists or histamine receptor antagonists, or any combination thereof.

60. A method of treating or preventing sepsis caused by S. pneumoniae infection in a subject, said method comprising administering a polypeptide of claim 52, or an analog, variant or fragment thereof, to said subject, in an amount sufficient to inhibit or abrogate sepsis in said subject.

61. The method according claim 60, wherein the subject is a human subject and wherein said subject is susceptible to S. pneumoniae infection, said subject is susceptible to, or is suffering from asthma, bronchospasm, bronchitis, bronchiolitis, and pneumonia, said subject is elderly or said subject is a child.

62. The method according to claim 60, wherein said human subject is further administered a decongestant, antibiotics, bronchodilators, anti-inflammatory steroids, leukotriene antagonists or histamine receptor antagonists, or any combination thereof.

63. A composition comprising:
a. an isolated human laminin alpha 5 polypeptide as set forth in SEQ ID NO 10;
b. an isolated nephronectin polypeptide consisting of an amino acid sequence as set forth in SEQ ID NOs 11-12;
c. an isolated SILV silver homolog polypeptide consisting of an amino acid sequence as set forth in SEQ ID NOs 13-14;
d. an isolated human SPOCK2 polypeptide as set forth in SEQ ID NO 15-17;
e. an isolated SPARC-like 1 polypeptide consisting of an amino acid sequence as set forth in SEQ ID NOs 18-19;
f. an isolated insulin-like growth factor 2 (somatomedin A) polypeptide consisting of an amino acid sequence as set forth in SEQ ID NO 20;
g. an isolated cadherin polypeptide consisting of an amino acid sequence as set forth in SEQ ID NOs 21-22;
h. an isolated desmocollin 2 precursor polypeptide consisting of an amino acid sequence as set forth in SEQ ID NO 23-24;
i. an isolated tectorin alpha polypeptide consisting of an amino acid sequence as set forth in SEQ ID NOs 25-26;
j. an isolated FRAS1 related extracellular matrix 1 polypeptide consisting of an amino acid sequence as set forth in SEQ ID NO 27;
k. an isolated metalloproteinase or metalloelastase polypeptide consisting of an amino acid sequence as set forth in SEQ ID NO 28;
l. an isolated carboxypeptidase Z polypeptide consisting of an amino acid sequence as set forth in SEQ ID NO 29-30;
m. an isolated lactrophilin 3 polypeptide consisting of an amino acid sequence as set forth in SEQ ID NO 31-32;
n. an isolated glycosyltransferase 25 domain polypeptide consisting of an amino acid sequence as set forth in SEQ ID NO 33-34;
or any combination thereof.

64. The composition of claim 63, wherein said composition further comprises one or more pharmaceutically active agents as decongestants, antibiotics, bronchodilators, anti-inflammatory steroids, leukotriene antagonists or histamine receptor antagonists, or a combination thereof.

65. The composition according to claim 63, wherein said composition is formulated for administration systemically, locally, by aerosol or topically.

66. A method of treating or preventing S. pneumoniae infection in a subject, said method comprising administering to said subject the composition of claim 63, wherein said polypeptide is in an amount sufficient to inhibit or abrogate S. pneumoniae infection.

67. The method according claim 66, wherein the subject is a human subject and wherein said subject is susceptible to S. pneumoniae infection, said subject is susceptible to, or is suffering from asthma, bronchospasm, bronchitis, bronchiolitis, and pneumonia, said subject is elderly or said subject is a child.

68. The method according to claim 66, wherein said human subject is farther administered a decongestant, antibiotics, bronchodilators, anti-inflammatory steroids, leukotriene antagonists or histamine receptor antagonists, or any combination thereof.

69. A method of treating or preventing sepsis caused by S. pneumoniae infection in a subject, said method comprising administering the composition of claim 63 to said subject, wherein said polypeptide, or an analog, variant or fragment thereof, is in an amount sufficient to inhibit or abrogate sepsis in said subject.

70. The method according claim 69, wherein the subject is a human subject and wherein said subject is susceptible to S. pneumoniae infection, said subject is susceptible to, or is suffering from asthma, bronchospasm, bronchitis, bronchiolitis, and pneumonia, said subject is elderly or said subject is a child.

71. The method according to claim 66, wherein said human subject is further administered a decongestant, antibiotics, bronchodilators, anti-inflammatory steroids, leukotriene antagonists or histamine receptor antagonists, or any combination thereof.

72. A method of treating or preventing S. pneumoniae infection in a subject, said method comprising administering to said subject:
a laminin alpha 5 protein,
a nephronectin protein,
a SILV silver homolog protein,
a sparc/osteonectin and SPOCK2 protein,
a SPARC-like 1 protein,
an insulin-like growth factor 2 (somatomedin A),
a cadherin protein,
a desmocollin 2 precursor protein,
a tectorin alpha protein,
a FRAS1 related extracellular matrix 1 protein,
a metalloproteinase or metalloelastase protein,
a carboxypeptidase Z protein,
a lactrophilin 3 protein,
or a glycosyltransferase 25 protein
or a combination thereof.

73. A method of treating or preventing sepsis caused by S. pneumoniae infection in a subject, said method comprising administering to said subject:
a laminin alpha 5 protein,
nephronectin protein,
a SILV silver homolog protein,
a sparc/osteonectin and SPOCK2 protein,
a SPARC-like 1 protein,
an insulin-like growth factor 2 (somatomedin A),
a cadherin protein,
a desmocollin 2 precursor protein,
a tectorin alpha protein,
a FRAS1 related extracellular matrix 1 protein,
a metalloproteinase or metalloelastase protein,
a carboxypeptidase Z protein,
a lactrophilin 3 protein,
or a glycosyltransferase 25 protein
or a combination thereof.
